(19) United States
(12) Patent Application Publication
(10) Pub. No.: US 2019/0231866 A1
(43) Pub. Date: Aug. 1, 2019

(54) METHODS FOR SAFE INDUCTION OF CROSS-CLADE IMMUNITY AGAINST HIV INFECTION IN HUMANS

(71) Applicant: Janssen Vaccines & Prevention B.V., Leiden (NL)

(72) Inventors: Maria Grazia PAU, Leiden (NL); Daniel John STIEH, Oegstgeest (NL); Frank TOMAKA, Titusville, NJ (US); Steven NIJS, Rotselaar (BE)

(21) Appl. No.: 16/255,987

(22) Filed: Jan. 24, 2019

Related U.S. Application Data
(60) Provisional application No. 62/622,186, filed on Jan. 26, 2018.

(51) Int. Cl. A61K 39/21; C12N 7/00

(52) U.S. Cl. CPC A61K 39/21; A61K 2039/5256; C12N 7/00

(57) ABSTRACT

Provided are means and methods for generating safe immune responses in humans against multiple clades of human immunodeficiency virus (HIV). The observed immune responses were improved over earlier reported immune responses in clinical trials.

### CROSS REFERENCE TO RELATED APPLICATION

This application claims priority under 35 U.S.C. § 119(e) to U.S. Provisional Patent Application No. 62/622,186, filed on Jan. 26, 2018, the disclosure of which is incorporated herein by reference in its entirety.

### STATEMENT REGARDING FEDERALLY SPONSORED RESEARCH OR DEVELOPMENT

This invention was made with government support under Grant No. AI096040 awarded by the National Institutes of Health. The government has certain rights in the invention.

### REFERENCE TO SEQUENCE LISTING SUBMITTED ELECTRONICALLY

This application contains a sequence listing, which is submitted electronically via EFS-Web as an ASCII formatted sequence listing with a file name “688097-438 (0310) Sequence Listing”, creation date of Jan. 23, 2019, and having a size of 61.8 kB. The sequence listing submitted via EFS-Web is part of the specification and is herein incorporated by reference in its entirety.

### BACKGROUND OF THE INVENTION

Human Immunodeficiency Virus (HIV) affects millions of people worldwide, and the prevention of HIV remains a very high priority, even in an era of widespread antiretroviral treatment. Despite the fact that the virus has been known for several decades now, there is still no preventive vaccine registered for use in humans. It is an urgent global health priority to find a safe and potent HIV vaccine that would prevent HIV infection. The ability of HIV to evade host immunity and constantly mutate makes development of a preventive vaccine a very challenging task. Such a development effort involves several steps, including performing studies that evaluate safety and immunogenicity of candidate components and compositions in humans, the outcome of which is unknowable before they are actually performed.

Clinical trials with adenovirus serotype 5 (Ad5) vectors in the past have shown disappointing results with respect to HIV vaccination, and even raised safety concerns for vaccines based on these vectors, especially for HIV vaccines (e.g. McElrath M J, et al., Lancet, 2008, 372: 1894-905).

Preclinical data have shown promising results with a strategy employing viral vectors encoding mosaic HIV antigens, designed to present a broader possible array of T cell epitopes to improve coverage of epitopes found in circulating HIV sequences (e.g., Fischer et al, Nat Med, 2007, 13: 100-6; Barouch et al, Cell, 2013, 155: 531-9), especially when combined in prime-boost regimens with adjuvanted HIV envelope (Env) protein (e.g., Barouch et al, Science, 2015, 349: 320-4; U.S. Provisional Application No. 62/535,458). Viral vectors used were replication-deficient adenovirus serotype 26 (Ad26) vectors. In certain experiments, the HIV envelope protein was a trimeric gp140 protein.

During the 9^{th} IAS conference on HIV science, held in July 2017, results from a phase 1/2a clinical trial were reported, wherein several prime-boost regimens using different viral vectors and optionally gp140 protein were tested in humans, and these studies showed promising results. It was reported that these vaccine regimens were immunogenic, and that there were about 100% responders showing humoral immunogenicity after the third vaccination in most groups, with a slight increase in ELISA titers observed in most groups that included gp140 protein in the boost. Cross-clade responses (e.g. against gp140 of clades A, B, consensus C, mosaic 1) were detected with very similar response patterns as to the vaccine clade C. Also good cellular immune responses were observed.

A particularly interesting regimen in these studies was a prime-boost regimen including as components three different mosaic antigens each encoded in a separate Ad26 vector, and an isolated trimeric clade C gp140 protein with aluminum phosphate (AlPO4) adjuvant, see e.g. U.S. Provisional Application No. 62/535,458. The three Ad26 vectors were in one composition and together encoded a mosaic version of a Gag-Pol fusion protein, a different mosaic version of a Gag-Pol fusion protein, and a mosaic version of an Env protein, hereinafter referred to as ‘trivalent Ad26’. The priming vaccination at weeks 0 and 12 contained the trivalent Ad26, while the boosting vaccinations at weeks 24 and 48 included the trivalent Ad26, as well as the adjuvanted trimeric clade C gp140 protein (in a separate composition, administered at the same time as the composition comprising the trivalent Ad26).

The vaccines tested in that study (herein referred to as the ‘APPROACH’ study, or ‘HIV-V-A004’) included one Ad26 encoding a mosaic Env antigen. In order to further broaden the immune responses it could potentially be beneficial to add another Env antigen to the vectors.

Recently, further Env antigen variants have been described (see WO 2017/102929, incorporated by reference herein), and a vector encoding such an antigen variant (e.g. a variant that will be referred to herein as ‘Mos2S.Env’ antigen), could potentially be a valuable addition to the trivalent Ad26 used in previous human studies.

However, no clinical data in humans have been reported so far for vectors encoding Mos2S.Env antigen. Safety, immune responses to this antigen, as well as any potential effects on immune responses to other components of a multi-component vaccine, are to be studied in the human population.

There remains a need in the art for further improved HIV vaccine candidates that generate safe and broad immune responses in humans.

### SUMMARY OF THE INVENTION

The invention is based on clinical trial findings in humans of a vaccine comprising a component consisting of Mos2S.Env antigen encoded by Ad26 (which component is referred to as ‘Ad26.Mos2S.Env’ herein). Such a vaccine appeared well-tolerated. The data show an unexpectedly significant increased magnitude of the humoral immune response in humans, as shown by ELISA binding of antibodies to Env proteins, when this vector is included together with the trivalent Ad26 composition tested before, as compared to the same regimen where Ad26.Mos2S.Env was not present. This increase was to autologous antigens, but also to Env of strains from clades not included in the vaccine. No antagonism of responses to the clade B responses most closely matched to the Ad26.Mos1.Env (one of the components of the trivalent Ad26 composition) was seen, which was unpredictable and took away a potential concern about addition of the Ad26.Mos2S.Env to the trivalent Ad26 composition when administered to humans. These data demonstrate that Ad26.Mos2S.Env can be safely added to an HIV vaccine regimen, and that this increases the magnitude of the immune response against HIV in humans. This brings the development of a safe and effective vaccine that could potentially prevent HIV in humans another important step further. A proof of concept study incorporating these components in a vaccine candidate, aimed at testing preventive efficacy of this vaccine candidate against HIV infection in a human population, has recently started (see e.g. www.imbokodo.org.za).

The invention provides a method of inducing a safe immune response against multiple clades of human immunodeficiency virus (HIV) in a human subject in need thereof, comprising:

(1) administering to the subject a priming composition comprising one or more Ad26 vectors together encoding at least the antigenic polypeptides having amino acid sequences that are at least 95%, at least 96%, at least 97%, at least 98%, at least 99% identical to SEQ ID NO: 3 and SEQ ID NO: 4 respectively, preferably wherein the encoded antigenic polypeptides have the amino acid sequences of SEQ ID NO: 3 and SEQ ID NO: 4 respectively, and a pharmaceutically acceptable carrier;
(2) administering to the subject, the priming composition;
(3) administering to the subject, a first boosting composition comprising at least one isolated HIV envelope glycoprotein, an adjuvant and a pharmaceutically acceptable carrier; and
(4) administering to the subject, together with (3), a second boosting composition comprising one or more Ad26 vectors together encoding at least the antigenic polypeptides having amino acid sequences that are at least 95%, at least 96%, at least 97%, at least 98%, at least 99% identical to SEQ ID NO: 3 and SEQ ID NO: 4 respectively, preferably wherein the encoded antigenic polypeptides have the amino acid sequences of SEQ ID NO: 3 and SEQ ID NO: 4 respectively, and a pharmaceutically acceptable carrier,
wherein the immune response comprises an antibody response that includes IgG that binds to isolated HIV envelope glycoproteins from strains of at least clades A, B, and C, when measured in enzyme-linked immunosorbent assays (ELISAs).

In certain embodiments, the method further comprises after step (4):

(5) administering to the subject, the first boosting composition; and
(6) administering to the subject, together with (5), the second boosting composition.

In certain embodiments, the priming composition and second boosting composition further comprise one or more Ad26 vectors together encoding the antigenic polypeptides having amino acid sequences that are at least 95%, at least 96%, at least 97%, at least 98%, at least 99% identical to SEQ ID NO: 1 and SEQ ID NO: 2 respectively, preferably wherein the encoded antigenic polypeptides have the amino acid sequences of SEQ ID NO: 1 and SEQ ID NO: 2.

In preferred embodiments, the at least one isolated HIV envelope glycoprotein in the first boosting composition has an amino acid sequence that is at least 95%, at least 96%, at least 97%, at least 98%, at least 99% identical to SEQ ID NO: 5. In a particularly preferred embodiment, said at least one isolated HIV envelope glycoprotein in the first boosting composition has the amino acid sequence of SEQ ID NO: 5.

In certain embodiments, the adjuvant in the first boosting composition is aluminium phosphate.

In certain embodiments, in each step wherein the Ad26 vectors are administered, these are administered at a total dose of about 5×10⁹ to about 1×10¹¹ vp, preferably about 5×10¹⁰ vp, of the Ad26 vectors.

In certain embodiments, in each step wherein the isolated HIV envelope glycoprotein is administered, this is administered at a total dose of about 125 μg to 350 μg, preferably about 250 μg.

In certain embodiments, step (2) is performed at about 10-14 weeks after step (1), steps (3) and (4) are performed at about 22-26 weeks after step (1). In case steps (5) and (6) are performed, in certain embodiments these are performed at about 42-60 weeks after step (1).

In certain embodiments, the priming composition and the second boosting composition each comprise a first Ad26 vector encoding SEQ ID NO: 1, a second Ad26 vector encoding SEQ ID NO: 2, a third Ad26 vector encoding SEQ ID NO: 3, and a fourth Ad26 vector encoding SEQ ID NO: 4. Preferably the priming composition and the second boosting composition are identical.

Preferably, the antibody response generated by the method as observed in a group of humans has a response rate of at least 90%, preferably at least 95%, most preferably about 100%.

In certain embodiments, the human subject is not infected with HIV when step (1) of the method is performed on the subject. Preferably, the human subject is not infected with HIV throughout the method.

Preferably, the antibody response is higher in magnitude, e.g. at least 1.5 fold, at least 2 fold, at least 2.5 fold, at least 3 fold, or at least 3.5 fold, e.g. about 1.8-6.7 fold, to isolated HIV envelope glycoprotein of at least a clade C strain, as compared to the same vaccine regimen wherein an Ad26 vector encoding an antigen having at least 95% identity to SEQ ID NO: 4, preferably having SEQ ID NO: 4, is replaced by an Ad26 vector encoding an antigen having at least 95% identity to SEQ ID NO: 3, preferably having SEQ ID NO: 3 (i.e., wherein twice the amount of Ad26 vector encoding SEQ ID NO: 3 is present as compared to the amount of Ad26 vector encoding SEQ ID NO: 3 in a method wherein Ad26 vector encoding SEQ ID NO: 3 and Ad26 vector encoding SEQ ID NO: 4 are both present). Preferably the antibody response in the method is higher in magnitude to isolated HIV Env glycoprotein of to at least a clade A, a clade B, and a clade C strain, as compared to the same vaccine regimen wherein an Ad26 vector encoding SEQ ID NO: 4 is replaced by an Ad26 vector encoding SEQ ID NO: 3.

In certain embodiments, step (2) of the method is performed about 12 weeks after step (1), and steps (3) and (4) are performed about 24 weeks after step (1).

In certain embodiments, steps (5) and (6) of the method are performed about 48 weeks after step (1).

In certain embodiments, in each respective step wherein the Ad26 vectors are administered, these are administered at a total dose of about 5×10¹⁰ vp.

In certain embodiments, in each step wherein the isolated HIV envelope glycoprotein is administered, this is administered at a dose of about 250 μg.

In certain embodiments, in each step wherein the isolated HIV envelope glycoprotein is administered, aluminium phosphate is administered at a dose of about 0.1-1.0 mg, e.g. about 0.425 mg aluminium.

In certain embodiments, the human subject to which the compositions are administered according to the invention, resides in an area or country where the predominant clade for HIV infections in humans is Clade A, Clade B, or Clade C, or a circulating recombinant form (CRF) derived from recombination between different clades of which at least one is Clade A, Clade B, or Clade C.

The invention also relates to a vaccine combination for use in inducing a safe immune response against multiple clades of HIV in a human subject in need thereof, wherein the vaccine combination comprises a primer vaccine and a booster vaccine according to embodiments of the invention. The invention also provides the use of a vaccine combination in the manufacture of a medicament for inducing a safe immune response against HIV in a human subject in need thereof, wherein the vaccine combination comprises a primer vaccine and a booster vaccine according to embodiments of the invention. All aspects and embodiments of the invention as described herein with respect to methods of inducing a safe immune response against HIV in a human subject can be applied to the vaccine combinations for use and/or uses of the vaccine combination in the manufacture of a medicament for inducing a safe immune response against HIV in a human subject in need thereof.

### BRIEF DESCRIPTION OF THE FIGURES

FIG. 1 shows data from clinical trial HPX2004 for IgG gp140 ENV ELISA clade C (C97ZA.012); data shown are from interim analysis at weeks 16 and 28 (i.e. four weeks after second and third vaccination, respectively);

FIG. 2 shows magnitude-breadth curve; 4 weeks after the 3rd vaccination, antibody response rates and magnitudes to 5 gp140 antigens were tested; the height of the lines indicates the proportion of study participants with levels of antibody binding across the x-axis and

FIG. 3 shows magnitude-breadth curve; 4 weeks after the 4th vaccination, antibody response rates and magnitudes to 5 gp140 antigens were tested; the height of the lines indicates the proportion of study participants with levels of antibody binding across the x-axis.

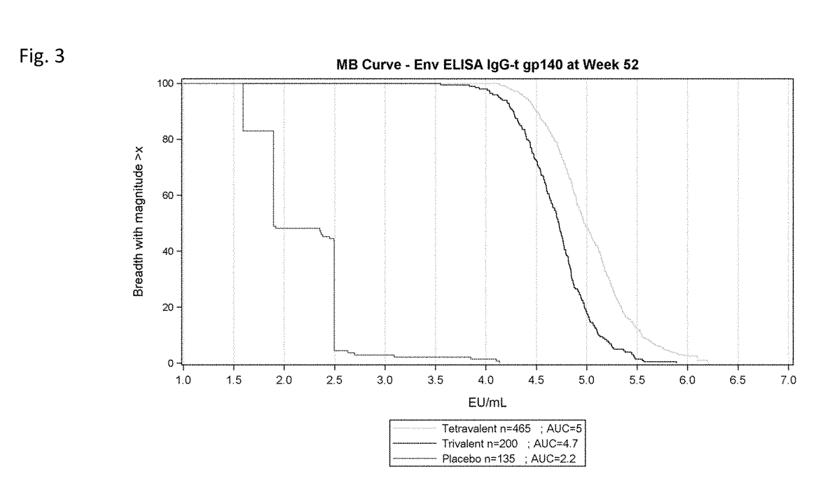

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

As used herein and in the appended claims, the singular forms “a,” “an,” and “the” include plural reference unless the context clearly dictates otherwise.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the invention can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

The term “percent (%) sequence identity” or “% identity” describes the number of matches (“hits”) of identical amino acids of two or more aligned amino acid sequences as compared to the number of amino acid residues making up the overall length of the amino acid sequences. In other terms, using an alignment, for two or more sequences the percentage of amino acid residues that are the same (e.g. 95%, 97% or 98% identity) may be determined, when the sequences are compared and aligned for maximum correspondence as measured using a sequence comparison algorithm as known in the art, or when manually aligned and visually inspected. The sequences which are compared to determine sequence identity may thus differ by substitution(s), addition(s) or deletion(s) of amino acids. Suitable programs for aligning protein sequences are known to the skilled person. The percentage sequence identity of protein sequences can, for example, be determined with programs such as CLUSTALW, Clustal Omega, FASTA or BLAST, e.g using the NCBI BLAST algorithm (Altschul S F, et al (1997), Nucleic Acids Res. 25:3389-3402).

As used herein, “subject” means any animal, preferably a mammal, most preferably a human, whom will be or has been subjected to a method according to an embodiment of the invention.

In certain embodiments, the human subject is a male. In certain embodiments, the human subject is a female. In certain embodiments, the human is at least 9 years old, at least 10, 11, 12, 13, 14, 15, 16, 17, 18, 19, 20, 21, 25, 30, 35 years old. In certain embodiments, the human subject is not older than 100 years, not older than 90 years, not older than 85 years, not older than 80 years, not older than 75 years, not older than 70 years, not older than 65 years, not older than 60 years, not older than 55 years, not older than 50 years. In certain embodiments, the human subject is between 9 and 65 years old. In certain embodiments, the human subject is between 18 and 50 years old. In preferred embodiments, the human subject does not have an HIV infection at least at the moment the priming composition is administered to said subject for the first time.

In certain embodiments, the human subject resides in an area or country where the predominant clade for HIV infections in humans is Clade A, Clade B, or Clade C, or a circulating recombinant form (CRF) derived from recombination between different clades of which at least one is Clade A, Clade B, or Clade C. Non-limiting examples of such CRFs are AE, AG, AB, BC, BG, BF, CD, etc. In certain embodiments, the predominant clade in such area or country is Clade A, or a CRF derived from recombination between different clades of which at least one is Clade A. In certain embodiments, the predominant clade in such area or country is Clade B, or a CRF derived from recombination between different clades of which at least one is Clade B. In certain embodiments, the predominant clade in such area or country is Clade C, or a CRF derived from recombination between different clades of which at least one is Clade C. A ‘predominant clade for HIV infections’ as used herein means the clade or CRF that of the HIV infections in the specific region or country is the most common clade or CRF found responsible for infections of humans in that region or country. Such clades or CRFs are determined by epidemiology testing (see e.g., Hemelaar J, 2012, Trends in Mol. Medicine 18(3): 182-192), and it is generally known which clades or CRFs are most common in specific regions or countries. For example, clade A is common in West Africa and Russia. Clade B is the dominant form in Europe, the Americas, and Japan, and is also the most common form in the Middle East and North Africa. Clade C is the dominant form in Southern Africa, Eastern Africa, India, and Nepal. In Australia, many new infections appear to be with Clade B, while most infections overall are with Clade C. In China, Clade C has been seen to be responsible for part of the HIV infections, but Clades/CRFs B, BC and AE appear predominant. The methods of the invention have been found to be particularly useful in that they provide strong antibody responses against Clade A, Clade B, and Clade C HIV, and hence can be used in vaccinations in regions or countries where any of these Clades, or CRFs derived at least in part from any of these Clades, are the predominant Clade, e.g. in any of the regions or countries mentioned above. In view of the broad antibody responses against at least HIV Clade A, B, and C shown herein, it is plausible that the methods of the invention can also be used in regions where other Clades or CRFs of HIV are predominant.

As used herein, the term “infection” refers to the invasion of a host by a disease causing agent, in this case the HIV virus.

As used herein, “a method of inducing safe and effective immune response” or “a safe method of inducing an effective immune response” means a method to induce an immune response that is effective to provide benefits of a vaccine, without causing unacceptable vaccine related adverse events, when administered to the human subject. Benefits of a vaccine include raising an immune response in a subject, which immune response may potentially lower the chance for future infection by HIV in the subject, or may potentially lower the detrimental effects of a future HIV infection in the subject, e.g. lower or prevent spreading of the virus in and/or out of the subject, and/or lower, delay, or prevent symptoms of HIV-induced disease.

As used herein, the phrase “unacceptable vaccine related adverse events,” “unacceptable adverse events,” and “unacceptable adverse reaction,” shall all mean harm or undesired outcome associated with or caused by the administration of a vaccine, and the harm or undesired outcome reaches such a severity that a regulatory agency deems the vaccine unacceptable for the proposed use. A “safe” immune response as used herein implicates that no unacceptable vaccine related adverse events have been observed in humans to a level that further administration of the vaccine component or vaccine components to humans would be permanently no longer allowed by regulatory authorities in the United States for safety reasons. Preferably, a safe immune response implies that administration of the vaccine components or vaccine for further clinical trials and/or (future) marketing is not permanently banned for safety reasons by regulatory authorities in other countries than the United States, e.g. countries from Europe, Africa, Asia, South America, Australia, and/or North America.

As used herein, an “effective immune response” or an “immune response” refers to an immune response that includes generation of antibodies to HIV envelope protein, which potentially could contribute to the mitigation or prevention of HIV infection in a human subject. Examples of effective immune responses include, but are not limited to, a humoral immunogenicity against HIV, such as an ADCP response to an isolated HIV envelope glycoprotein, IgG binding to HIV envelope glycoproteins as measured by ELISA, a cellular immune response as measured by a IFNγ response in an ELISPOT to a potential T-cell epitopes (PTE) peptide pool, etc. An “effective immune response” can but does not necessarily refer to protective immunity in a human subject. Preferably, an effective immune response induced in a vaccinated human subject includes generation of antibodies that recognize HIV envelope protein from strains of different clades, including clade C and clade B, and preferably clade A. Preferably such an effective immune response is induced in at least 90% (i.e. the response rate is at least 90%), 91%, 92%, 93%, 94%, 95%, 96%, 97%, 98%, 99%, preferably in about 100% of the vaccinated human subjects. More preferably, the level of such antibodies generated against at least clade C and clade B on average is substantially higher in subjects vaccinated according to the methods of the present invention as compared to subjects vaccinated according to the same protocol wherein the Ad26 encoding SEQ ID NO: 4 is replaced with Ad26 encoding SEQ ID NO: 3.

As used herein, a “response rate” refers to the number of subjects who have responded to a treatment with a particular outcome divided by the number of treated subjects. As used herein, a “potential T-cell epitopes (PTE) peptide pool” refers to a pool of peptides containing potential T-cell epitope (PTE) peptides embedded in antigenic protein sequences of circulating strains of HIV-1 worldwide. Examples of a “potential T-cell epitopes (PTE) peptide pool” include, but are not limited to, a HIV-1 PTE Gag peptide pool, a HIV-1 PTE Env peptide pool, and a HIV-1 PTE Pol peptide pool, which are available from the U.S. National Institute of Health AIDs Reagent Program.

Human immunodeficiency virus (HIV) is a member of the genus Lentivirinae, which is part of the family of Retroviridae. Two species of HIV infect humans: HIV-1 and HIV-2. HIV-1 is the most common strain of HIV virus, and is known to be more pathogenic than HIV-2. As used herein, the terms “human immunodeficiency virus” and “HIV” refer, but are not limited to, HIV-1 and HIV-2, preferably HIV-1.

HIV is categorized into multiple clades with a high degree of genetic divergence. As used herein, the term “HIV clade” or “HIV subtype” refers to related human immunodeficiency viruses classified according to their degree of genetic similarity. There are currently three groups of HIV-1 isolates: M, N and O. Group M (major strains) consists of at least ten clades, A through J. Group O (outer strains) can consist of a similar number of clades. Group N is a new HIV-1 isolate that has not been categorized in either group M or O. In certain exemplary embodiments, a broadly neutralizing antibody described herein will recognize and raise an immune response against two, three, four, five, six, seven, eight, nine, ten or more clades and/or two or more groups of HIV. In preferred embodiments, the methods of the invention generate antibody responses in the vaccinated human subject, which antibodies include at least antibodies against strains from clade C, and antibodies against clade B, as can be measured by ELISA.

Antigenic HIV Polypeptides

As used herein, the term “antigenic polypeptide of an HIV,” “HIV antigenic polypeptide,” “HIV antigenic protein,” “HIV immunogenic polypeptide,” or “HIV immunogen” or “HIV antigen” refers to a polypeptide capable of inducing an immune response, e.g., a humoral and/or cellular mediated response, against HIV in a subject in need thereof. The antigenic polypeptide can be a protein of the HIV, a fragment or epitope thereof, or a combination of multiple HIV proteins or portions thereof, that can induce an immune response or produce an immunity against the HIV in a subject in need thereof.

According to embodiments of the invention, the antigenic polypeptide can be an HIV-1 antigen or fragments thereof. Examples of HIV antigens include, but are not limited to gag, pol, and env gene products, which encode structural proteins and essential enzymes. Gag, pol, and env gene products are synthesized as polyproteins, which are further processed into multiple other protein products. The primary protein product of the gag gene is the viral structural protein gag polyprotein, which is further processed into MA, CA, SP1, NC, SP2, and P6 protein products. The pol gene encodes viral enzymes (Pol, polymerase), and the primary protein product is further processed into RT, RNase H, IN, and PR protein products. The env gene encodes structural proteins, specifically glycoproteins of the virion envelope. The primary protein product of the env gene is gp160, which is further processed into gp120 and gp41.

According to a preferred embodiment, the antigenic polypeptide comprises an HIV-1 Gag, Env, or Pol antigen, or any portion or combination thereof.

Mosaic Antigens

A mosaic HIV antigen according to the invention is preferably a mosaic HIV-1 Gag, Pol, or Env antigen. As used herein, “a mosaic HIV Gag, Pol, or Env antigen” specifically refers to a mosaic antigen comprising multiple epitopes derived from one or more of the Gag, Pol and Env polyprotein sequences of HIV. The epitope sequences of the mosaic HIV Gag, Pol, or Env antigens according to the invention resemble the sequences of the natural HIV antigens, but are optimized to present a broader possible array of T cell epitopes to improve coverage of epitopes found in circulating HIV sequences.

For example, to provide maximal coverage of potential T-cell epitopes, mosaic Gag, Pol and Env antigens are designed to provide optimal coverage of one or more HIV clades. Sequence Database in silico recombinant sequences of fragments of 9 contiguous amino acids (9-mers) are selected that resemble real proteins and that maximize the number of 9-mer sequence matches between vaccine candidates and the global database. The mosaic Gag, Pol and Env antigens have similar domain structure to natural antigens and consist entirely of natural sequences with no artificial junctions. The Pol antigens can contain mutants to eliminate catalytic activity. The monomeric Env gp140 mosaic antigens can contain point mutations to eliminate cleavage and fusion activity.

In one embodiment, a mosaic HIV Gag, Pol, or Env antigen according to the invention is a mosaic HIV Gag antigen with epitopes derived from the sequences of gag gene products; a mosaic HIV Pol antigen with epitopes derived from the sequences of pol gene products; or a mosaic HIV Env antigen with epitopes derived from the sequences of env gene products.

In certain embodiments, a mosaic HIV Gag, Pol, or Env antigen according to the invention comprises a combination of epitopes derived from sequences of gag, pol, and/or env gene products. Illustrative and non-limiting examples include mosaic Gag-Pol antigens with epitopes derived from the sequences of gag and pol gene products.

Preferably, mosaic HIV Gag, Pol, or Env antigens include, but are not limited to, antigens comprising the amino acid sequences selected from the group consisting of SEQ ID NOs: 1-4. Preferably, such mosaic antigens are encoded by vectors, e.g. adenoviral vectors, such as Ad26 vectors.

In view of the present disclosure, a mosaic HIV antigen or vectors encoding such can be produced using methods known in the art. See, for example, US20120076812, Fischer et al, Nat Med, 2007. 13(1): p. 100-6; Barouch et al., Nat Med 2010, 16:319-323.

Adenovirus Vectors

As used herein, the notation “rAd” means recombinant adenovirus, e.g., “rAd26” refers to recombinant human adenovirus serotype 26. According to the methods of the invention, an adenovirus is a human adenovirus serotype 26. An advantage of rAd26 is a low seroprevalence and/or low pre-existing neutralizing antibody titers in the human population. Preparation of recombinant adenoviral vectors is well known in the art, and preparation of rAd26 vectors is described, for example, in WO 2007/104792 and in Abbink et al., (2007) Virol 81(9): 4654-63. Exemplary genome sequences of Ad26 are found in GenBank Accession EF 153474 and in SEQ ID NO: 1 of WO 2007/104792.

An “adenovirus capsid protein” refers to a protein on the capsid of an adenovirus (e.g., Ad26 vectors) that is involved in determining the serotype and/or tropism of a particular adenovirus. Adenoviral capsid proteins typically include the fiber, penton and/or hexon proteins. As used herein a “capsid protein” for a particular adenovirus, such as an “Ad26 capsid protein” can be, for example, a chimeric capsid protein that includes at least a part of an Ad26 capsid protein. In certain embodiments, the capsid protein is an entire capsid protein of Ad26. In certain embodiments, the hexon, penton and fiber are of Ad26. Thus, the vectors that can be used in the invention comprise an Ad26 capsid protein (e.g., a fiber, penton or hexon protein). One of ordinary skill in the art will recognize that it is not necessary that an entire Ad26 capsid protein be used in the vectors of the invention. In preferred embodiments, the fiber, penton and hexon proteins are each derived from Ad26.

In preferred embodiments, the recombinant adenovirus vector useful in the invention is derived mainly or entirely from Ad26 (i.e., the vector is rAd26). In preferred embodiments, the adenovirus is replication deficient, e.g., because it contains a deletion in the E1 region of the genome. For the adenoviruses of the invention, being derived from Ad26, it is typical to exchange the E4-orf6 coding sequence of the adenovirus with the E4-orf6 of an adenovirus of human subgroup C such as Ad5. This allows propagation of such adenoviruses in well-known complementing cell lines that express the E1 genes of Ad5, such as for example 293 cells, PER.C6 cells, and the like (see, e.g. WO 03/104467). However, such adenoviruses will not be capable of replicating in non-complementing cells that do not express the E1 genes of Ad5, nor in human hosts that are vaccinated with such adenoviruses.

In certain embodiments, the adenovirus is a human adenovirus of serotype 26, with a deletion in the E1 region into which the nucleic acid encoding the one or more mosaic HIV antigenic polypeptides has been cloned, and with an E4 orf6 region of Ad5. In an embodiment of the invention, some vectors useful for the invention include those described in WO2012/082918 and in WO 2017/102929.

Typically, a vector useful in the invention is produced using a nucleic acid comprising the entire recombinant adenoviral genome (e.g., a plasmid, cosmid, or baculovirus vector). The nucleic acid molecules can be in the form of RNA or in the form of DNA obtained by cloning or produced synthetically. The DNA can be double-stranded or single-stranded.

In some embodiments, the vectors of the invention can contain deletions in other regions than E1, such as the E2, E3 or E4 regions, or insertions of heterologous genes linked to a promoter within one or more of these regions. For E2- and/or E4-mutated adenoviruses, generally E2- and/or E4-complementing cell lines are used to generate recombinant adenoviruses. Mutations in the E3 region of the adenovirus need not be complemented by the cell line, since E3 is not required for replication.

A packaging cell line is typically used to produce sufficient amounts of adenovirus vectors for use in the invention. A packaging cell is a cell that comprises those genes that have been deleted or inactivated in a replication deficient vector, such as E1, thus allowing the virus to replicate in the cell. Suitable packaging cell lines that are known in the art include, for example, PER.C6, 911, 293, and E1 A549.

If required, the heterologous gene encoding the HIV antigenic polypeptides in the adenovirus vectors can be codon-optimized to ensure proper expression in the treated host (e.g., human). Codon-optimization is a technology widely applied in the art. Typically, the heterologous gene is cloned into the E1 and/or the E3 region of the adenoviral genome. The skilled person is well aware of producing different nucleic acid sequences that encode a protein, in view of the redundancy of the genetic code. Different variants of Ad26 vectors encoding a specified protein sequence, by using variant coding sequences that encode the same specified protein, may thus be prepared according to routine methods in view of the present disclosure and common general knowledge of the skilled person. Any Ad26 vector that includes a nucleic acid that encodes a specified HIV antigen amino acid sequence can therefore be used in the invention. Non-limiting examples of suitable nucleic acid sequences that can be used to encode SEQ ID NOs 1-4, respectively, in adenovirus vectors that can be used in the priming and/or second boosting composition, are provided as SEQ ID NOs: 7-10, respectively.

The heterologous HIV gene encoded in the adenovirus vector can be under the control of (i.e., operably linked to) an adenovirus-derived promoter (e.g., the Major Late Promoter), or can be under the control of a heterologous promoter. Examples of suitable heterologous promoters include the cytomegalovirus (CMV) promoter and the Rous Sarcoma virus (RSV) promoter. Preferably, the promoter is located upstream of the heterologous gene of interest within an expression cassette. In certain embodiments, a CMV promoter is used to drive expression of the HIV antigens in the Ad26 vectors that can be used according to the invention.

As noted above, the adenovirus vectors useful for the invention can encode a wide variety of HIV antigenic polypeptides known to those of skill in the art, including but not limited to, the antigenic polypeptides discussed herein. In preferred embodiments the one or more rAd26 vectors together encode HIV antigenic polypeptides having amino acid sequences of SEQ ID NOs: 1, 2, 3, and 4.

According to embodiments of the invention, the first composition can comprise one rAd26 vector, or more than one rAd26 vector. In certain embodiments, the first composition comprises one, two, three, or four, etc. rAd26 vectors. The one or more rAd26 vectors can express the same or different HIV antigenic polypeptides. Each of the vectors can express one HIV antigenic polypeptide sequence, or more than one HIV antigenic polypeptide sequence. As an illustrative and non-limiting preferred example, the first composition can comprise four rAd26 vectors, each expressing a different HIV antigenic polypeptide, preferably SEQ ID NOs: 1, 2, 3, and 4, respectively.

Envelope Glycoprotein

As used herein, each of the terms “envelope glycoprotein,” “env glycoprotein,” “isolated HIV envelope glycoprotein”, and “Env” refers to, but is not limited to, the glycoprotein that is expressed on the surface of the envelope of HIV virions and the surface of the plasma membrane of HIV infected cells, or a fragment thereof that can induce an immune response or produce an immunity against the HIV in a subject in need thereof. In certain embodiments, the isolated HIV envelope glycoprotein is a recombinantly produced variant, e.g. a stabilized trimeric gp140 protein. The term “isolated” herein refers to Env protein that is outside of the context of an HIV viral particle, and preferably refers to an Env protein preparation that is at least 50% pure, i.e. it includes less than 50% of non-Env proteins, preferably at least 70%, 80%, 90%, 95% pure. Preferably it is obtained by recombinant expression.

The env gene of HIV encodes gp160, which is proteolytically cleaved into gp120 and gp41. More specifically, gp160 trimerizes to (gp160)₃ and then undergoes cleavage into the two noncovalently associated fragments gp120 and gp41. Viral entry is subsequently mediated by a trimer of gp120/gp41 heterodimers. Gp120 is the receptor binding fragment, and binds to the CD4 receptor on a target cell that has such a receptor, such as, e.g., a T-helper cell. Gp41, which is non-covalently bound to gp120, is the fusion fragment and provides the second step by which HIV enters the cell. Gp41 is originally buried within the viral envelope, but when gp120 binds to a CD4 receptor, gp120 changes its conformation causing gp41 to become exposed, where it can assist in fusion with the host cell. Gp140 is the uncleaved ectodomain of trimeric gp160, i.e., (gp160)₃, that has been used as a surrogate for the native state of the cleaved, viral spike.

According to embodiments of the invention, env glycoproteins (e.g, gp160, gp140, gp120, or gp41), preferably stabilized trimeric gp140 protein, can be administered for boosting immunizations to enhance the immunity induced by expression vectors alone.

As used herein, each of the terms “stabilized trimeric gp140 protein” and “stabilized trimer of gp140” refers to a trimer of gp140 polypeptides that includes a polypeptide sequence that increases the stability of the trimeric structure, e.g. a trimerization domain that stabilizes trimers of gp140. Examples of trimerization domains include, but are not limited to, the T4-fibritin “foldon” trimerization domain; the coiled-coil trimerization domain derived from GCN4; and the catalytic subunit of E. coli aspartate transcarbamoylase as a trimer tag.

According to one embodiment of the invention, isolated HIV envelope glycoprotein, preferably in the form of a stabilized trimeric gp140 protein, can be administered as a boosting immunization or as a component of a boosting immunization together with viral expression vectors. Preferably, the stabilized trimeric gp140 protein is a clade C gp140 protein.

In a particular embodiment of the invention, a stabilized trimeric gp140 protein comprises the amino acid sequence of SEQ ID NO: 5 (clade C gp140 protein). This protein includes a foldon trimerization domain. Other forms of isolated HIV envelope glycoprotein could alternatively or additionally be used in the first boosting composition of the invention, e.g. AIDSVAX B/E, produced in genetically engineered CHO cells, is a bivalent HIV gp120 envelope glycoprotein vaccine containing a CRF01_AE envelope from the HIV-1 strain A244 and a subtype B envelope from the HIV-1 strain MN, and has been used in partly successful clinical trials before. Trimeric Env proteins are however preferred.

A preferred dose for the total amount of isolated HIV envelope glycoprotein, e.g. trimeric gp140 protein, for administration to humans is between about 125 and 350 μg, preferably about 250 μg. If two different variants of isolated HIV envelope glycoprotein are administered, a suitable dose would for instance be about 125 μg of each glycoprotein, to a total of 250 μg of isolated HIV envelope glycoprotein for an administration to humans.

As used herein, the term “co-delivery” or “administered together with” refers to simultaneous administration of two components, such as a viral expression vector and an isolated antigenic polypeptide. “Simultaneous administration” can be administration of the two components at least within the same day. When two components are “administered together with,” they can be administered in separate compositions sequentially within a short time period, such as 24, 20, 16, 12, 8 or 4 hours, or within 1 hour, or within 5 minutes or they can be administered in a single composition or in multiple compositions at the same time or essentially at the same time.

An isolated HIV envelope glycoprotein can be co-delivered with an adenovirus (e.g. Ad26) expression vector. According to a preferred embodiment, isolated HIV envelope glycoprotein and Ad26 are administered separately, as two distinct formulations. Alternatively, isolated HIV envelope glycoprotein protein can be administered with Ad26 together in a single formulation. Furthermore, an isolated HIV envelope glycoprotein protein can be administered in an adjuvanted formulation. In one embodiment, aluminum phosphate is used as adjuvant for Env protein.

Antigenic polypeptides can be produced and isolated using any method known in the art in view of the present disclosure (see e.g. Nkolola et al 2010, J. Virology 84(7): 3270-3279; Kovacs et al, PNAS 2012, 109(30):12111-6, WO 2010/042942 and WO 2014/107744). For example, an antigenic polypeptide can be expressed from a host cell, preferably a recombinant host cell optimized for production of the antigenic polypeptide. According to certain embodiments, a recombinant gene is used to express a gp140 protein containing mutations to eliminate cleavage and fusion activity. The gp140 protein can also include cleavage site mutation(s), a factor Xa site, and/or a foldon trimerization domain. A leader/signal sequence can be operably linked to the N-terminal of an optimized gp140 protein for maximal protein expression. The leader/signal sequence is usually cleaved from the nascent polypeptide during transport into the lumen of the endoplasmic reticulum. Any leader/signal sequence suitable for a host cell of interest can be used. A non-limiting example of a leader/signal sequence comprises the amino acid sequence of SEQ ID NO: 6. A non-limiting example of a sequence encoding trimeric gp140 protein, that can be used to recombinantly express this protein in eukaryotic, e.g. mammalian host cells, is provided as SEQ ID NO: 11. Upon expression, the gp140 protein can be harvested and purified from the host cells, and used as isolated HIV envelope glycoprotein in a first boosting vaccine composition according to the invention.

Immunogenic Compositions

As used herein, “an immunogenically effective amount” or “immunologically effective amount” means an amount of a composition sufficient to induce a safe and effective immune response in a human subject in need thereof.

It is possible to administer an immunogenically effective amount to a subject, and subsequently administer another dose of an immunogenically effective amount to the same subject, in a so-called prime-boost regimen. This general concept of a prime-boost regimen is well known to the skilled person in the vaccine field. Further booster administrations can optionally be added to the regimen, as needed.

An immunogenically effective amount can be administered in a single step (such as a single injection), or multiple steps (such as multiple injection), or in a single composition or multiple compositions.

Immunogenic compositions are compositions comprising an immunogenically effective amount of purified or partially purified adenovirus vectors or isolated HIV envelope glycoprotein for use in the invention. Said compositions can be formulated as a vaccine (also referred to as an “immunogenic composition”) according to methods well known in the art. Such compositions can include adjuvants to enhance immune responses. The optimal ratios of each component in the formulation can be determined by techniques well known to those skilled in the art in view of the present disclosure.

The compositions of the invention can optionally comprise other HIV-1 antigens or the priming or boosting immunizations can optionally comprise other antigens. The other antigens that optionally can be used in combination with the adenovirus vectors of the invention are not critical to the invention and can be, for example, HIV-1 antigens and nucleic acids expressing them.

The compositions of the invention can comprise a pharmaceutically acceptable excipient, carrier, buffer, stabilizer or other materials well known to those skilled in the art. Such materials should be non-toxic and should not interfere with the efficacy of the active ingredient, and are referred to herein as ‘pharmaceutically acceptable carrier’. The precise nature of the carrier or other material can depend on the route of administration, e.g., intramuscular, subcutaneous, oral, intravenous, cutaneous, intramucosal (e.g., gut), intranasal or intraperitoneal routes. In certain embodiments, the methods of the invention comprise intramuscular injection of the immunogenic compositions.

According to embodiments of the invention, upon administration to a subject, an expression vector, such as a recombinant adenovirus vector, expresses an immunogenic polypeptide. The expressed immunogenic polypeptide is presented to the immune system of the subject, thereby inducing the required response to produce immunity, or induce an immune response that may help in preventing a disease or infection. For example, the response can be the production of antibodies specific to the immunogenic polypeptide.

Preferably, upon administration to a subject, a rAd26 vector expresses a mosaic HIV Gag, Pol, or Env antigen. Presentation of a mosaic HIV Gag, Pol, and/or Env antigen according to the invention to the immune system of a subject can induce the production of antibodies specific to the HIV gag, pol, and/or env gene products, depending on the sequence composition of the expressed mosaic HIV antigen.

According to embodiments of the invention, an immunogenically effective amount of the priming composition or the second boosting composition when used with reference to total amount of Ad26 vectors in the composition can range from about 5×10⁹ to about 1×10¹¹ viral particles, for example 5×10⁹, 10¹⁰, 5×10¹⁰ or 10¹¹ viral particles. In certain embodiments, when 4 adenoviral vectors are present in a composition, they are present at a 1:1:1:1 ratio. Other ratios can also be used.

Typically, the Ad26 vectors are in pharmaceutically acceptable compositions. For instance, recombinant adenovirus vector may be stored in the buffer that is also used for the Adenovirus World Standard: 20 mM Tris pH 8, 25 mM NaCl, 2.5% glycerol. Another useful adenovirus formulation buffer suitable for administration to humans is 20 mM Tris, 2 mM MgCl₂, 25 mM NaCl, sucrose 10% (w/v), polysorbate-80 0.02% (w/v). Another suitable formulation for Ad26 that can be used in the present invention is 20 mM L-histidine, 75 mM NaCl, 5% (w/v) sucrose, 0.02% (w/w) polysorbate-80, 0.1 mM EDTA, 0.5% (v/v) ethanol. Another formulation buffer that is suitable for stable storage of the adenovirus at 2-8° C. and for administration to humans comprises 10-25 mM citrate buffer pH 5.9-6.2, 4-6% (w/w) hydroxypropyl-beta-cyclodextrin (HBCD), 70-100 mM NaCl, 0.018-0.035% (w/w) polysorbate-80, and optionally 0.3-0.45% (w/w) ethanol, e.g. 15 mM citrate buffer, 5% HBCD, 75 mM NaCl, 0.03% polysorbate-80, and 0.4% ethanol. Suitable compositions are typically prepared using water for injection in sufficient quantity to reach the indicated concentrations. Obviously, many other buffers can be used, and several examples of suitable formulations for the storage and for pharmaceutical administration of purified vectors are known. In exemplary non-limiting embodiments, the Ad26 vectors may be present in such formulations as 4 different Ad26 vectors (each having a different HIV antigen insert), each at a concentration of 2.5×10¹⁰ vp/mL, to a final concentration of the combined Ad26 vectors of 1×10¹¹ vp/mL.

According to embodiments of the invention, when used with reference to the total amount of the isolated HIV envelope glycoprotein in the first boosting composition, such as the isolated gp140 protein having the amino acid sequence of SEQ ID NO: 5, an immunogenically effective amount can range from, e.g. about 50 μg to 350 μg, e.g. about 50, 75, 100, 125, 150, 200, 250, 300, or 350 μg.

The terms “adjuvant” and “immune stimulant” are used interchangeably herein, and are defined as one or more substances that cause stimulation of the immune system. In this context, an adjuvant is used to enhance an immune response to the isolated HIV envelope glycoprotein. The immunogenic compositions useful in the invention can comprise adjuvants. Adjuvants suitable for co-administration in accordance with the invention should be ones that are potentially safe, well tolerated and effective in people, and non-limiting examples include QS-21, MPL, CpG, Aluminium salts (e.g. aluminum phosphate, e.g. AdjuPhos), and MF59.

In a preferred embodiment, the adjuvant is an aluminum salt, such as aluminum phosphate, e.g. AdjuPhos. In certain embodiments, the aluminum phosphate is preferably present in or administered with a composition with isolated HIV envelope glycoprotein, such as gp140. Preferably, isolated HIV envelope glycoprotein is co-formulated with aluminum phosphate in a single composition when this is administered to the human subject. Alternatively, the Env protein and the adjuvant may be in separate compositions that are co-delivered to the subject.

According to embodiments of the invention, when used with reference to the total amount of aluminum phosphate in a boosting composition comprising one or more HIV envelope polypeptides, the total amount of aluminum in the aluminum phosphate administered can range from, e.g. about 10 μg to about 1000 μg, e.g. about 200 μg to 650 μg, e.g. about 200, 250, 300, 350, 400, 425, 450, 475, 500, 550, or 600 μg, preferably about 425 μg of aluminum.

One non-limiting example of a suitable formulation for isolated HIV Env glycoprotein that can be used for the first boosting composition includes: 20 mM HEPES pH 6.5, 90 mM NaCl, 0.02% (w/v) polysorbate-80, 4% (w/v) sucrose. Another non-limiting example of a suitable formulation for isolated HIV envelope glycoprotein that can be used for the first boosting composition includes: 5-20 mM Histidine buffer pH 5.5-7.0, 0.01-0.05% (w/v) polysorbate-20, and 2-15% (w/v) sorbitol, e.g. 10 mM Histidine pH 6.5, 0.02% polysorbate-20, 12% sorbitol. In exemplary non-limiting embodiments, the isolated HIV envelope glycoprotein, e.g. stable trimeric gp140, e.g. having the amino acid sequence of SEQ ID NO: 5, may be present in a concentration of about 0.05 to 5.0 mg/mL, e.g. 0.5 mg/mL. In certain embodiments, aluminum phosphate is also present in the latter formulation, e.g. at a concentration of 0.7-4.0 mg/mL, e.g. 0.85 mg/mL. Such a formulation is stable at 2-8° C. for at least six months. One or more variants of an isolated HIV envelope glycoprotein may be present in such compositions.

The ability to induce or stimulate an anti-HIV immune response upon administration in an animal or human organism can be evaluated either in vitro or in vivo using a variety of assays which are standard in the art. Measurement of cellular immunity can be performed by measurement of cytokine profiles secreted by activated effector cells including those derived from CD4+ and CD8+ T-cells (e.g. quantification of IL-10 or IFN gamma-producing cells by ELISPOT), by determination of the activation status of immune effector cells (e.g. T cell proliferation assays by a classical [³H] thymidine uptake), by assaying for antigen-specific T lymphocytes in a sensitized subject (e.g. peptide-specific lysis in a cytotoxicity assay, etc.).

The ability to stimulate a cellular and/or a humoral response can be determined by antibody binding and/or competition in binding. For example, titers of antibodies produced in response to administration of a composition providing an immunogen can be measured by enzyme-linked immunosorbent assay (ELISA). The immune responses can also be measured by neutralizing antibody assay, where a neutralization of a virus is defined as the loss of infectivity through reaction/inhibition/neutralization of the virus with specific antibody. The immune response can further be measured by Antibody-Dependent Cellular Phagocytosis (ADCP) Assay.

Vaccine Combination

A vaccine combination useful for inducing an immune response against a human immunodeficiency virus (HIV) in a subject in need thereof according to the invention, can comprise:
(i) a priming composition comprising one or more Ad26 vectors together encoding HIV antigenic polypeptides having amino acid sequences that are at least 95%, preferably 100% identical to SEQ ID NO: 3 and SEQ ID NO: 4, respectively, and a pharmaceutically acceptable carrier;
(ii) a first boosting composition comprising an isolated HIV envelope glycoprotein, an adjuvant and a pharmaceutically acceptable carrier;
(iii) a second boosting composition comprising one or more Ad26 vectors together encoding HIV antigenic polypeptides having amino acid sequences that are at least 95%, preferably 100% identical to SEQ ID NO: 3 and SEQ ID NO: 4, respectively, and a pharmaceutically acceptable carrier.

Preferably the priming composition and second boosting composition further comprise one or more Ad26 vectors together encoding the antigenic polypeptides having amino acid sequences that are at least 95% identical to SEQ ID NO: 1 and SEQ ID NO: 2 respectively, and preferably the isolated HIV envelope glycoprotein in the first boosting composition has an amino acid sequence that is at least 95% identical, preferably 100% identical, to SEQ ID NO: 5. Other preferred features of the compositions are as indicated hereinabove.

Such vaccine combinations are effective to induce an immune response in humans against multiple clades of HIV.

Method for Inducing Immunity Against HIV

According to embodiments of the invention, “inducing an immune response” when used with reference to the methods described herein encompasses providing immunity and/or vaccinating a subject against an HIV infection, for prophylactic purposes. Preferably, the methods of the invention are for prophylactic purposes, such as for providing immunity that may help mitigating effects of HIV infection, potentially helping to prevent infection by HIV of the vaccinated human subject and/or potentially helping to prevent spreading of HIV by such subject into the population. Preferably the subject to which the compositions is administered is a human subject uninfected by HIV, at least at the moment of first administration of the priming vaccine composition, preferably at the moment of administration of any of the vaccine compositions used in the invention. Preferably the human subject is seronegative for HIV at the moment of the first administration of the priming vaccine composition.

In one embodiment of the disclosed methods, one or more rAd26 vectors together encoding the indicated HIV antigenic polypeptides having SEQ ID NOs: 3 and 4, preferably 1-4, respectively, are used to prime the immune response. One or more isolated HIV envelope glycoproteins can be used together with the one or more adenovirus vectors for the boosting immunization. The priming immunization can be administered multiple times, for example, initial priming administration at time 0, followed by another priming administration about 10-14 weeks, such as 10, 11, 12, 13 or 14 weeks, after the initial priming administration. One or more isolated HIV envelope glycoproteins together with one or more additional rAd26 vectors encoding the indicated HIV antigenic polypeptides are used to boost the immune response. The boosting immunization can also be administered multiple times, for example, first at about 22-26 weeks, such as 22, 23 24, 25, or 26 weeks, after the initial priming administration. In certain preferred embodiments, this is followed by another boosting administration at about 42-60 weeks, such as 42, 43, 44, 45, 46, 47, 48, 49, 50, 51, 52, 53, 54, 55, 56, 57, 58, 59 or 60 weeks after the initial priming administration. The immune response induced by the immunization can be monitored.

Embodiments of the disclosed methods also contemplate shorter prime-boost regimens, meaning that the final boosting immunization is administered about 22-26 weeks after the initial priming administration, and the priming immunization can be administered at week 0, and re-administered at about 10-14 weeks. The boosting immunization can also be administered multiple times following the priming administration.

It is readily appreciated by those skilled in the art that the regimen for the priming and boosting administrations can be adjusted based on the measured immune responses after the administrations. For example, the boosting compositions are generally administered weeks or months after administration of the priming composition, for example, about 2-3 weeks or 4 weeks, or 8 weeks, or 16 weeks, or 20 weeks, or 24 weeks, or 28 weeks, or 30 weeks or 32 weeks or one to two years after administration of the priming composition.

In certain embodiments, a first boosting immunization is administered 10-36 weeks after the last priming, more preferably 12-24 weeks after priming.

The antigens in the respective priming and boosting compositions (however many boosting compositions are employed) need not be identical, but preferably share antigenic determinants or be substantially similar to each other. In preferred embodiments, the priming composition and the second boosting composition are identical.

Administration of the immunogenic compositions comprising the Ad26 vectors and/or isolated HIV Env glycoprotein is typically intramuscular. However other modes of administration such as subcutaneous, intravenous, cutaneous, intradermal or nasal can be envisaged as well. Intramuscular administration of the immunogenic compositions can be achieved by using a needle to inject a suspension of the Ad26 vectors, and/or isolated HIV Env glycoprotein. An alternative is the use of a needleless injection device to administer the composition (using, e.g., Biojector™) or a freeze-dried powder containing the vaccine.

Typically, administration of the vaccine compositions according to embodiments of the invention will have a prophylactic aim to generate an immune response against an HIV antigen before infection or development of symptoms.

The compositions can, if desired, be presented in a kit, pack or dispenser, which can contain one or more unit dosage forms containing the active ingredients. The kit, for example, can comprise metal or plastic foil, such as a blister pack. The kit, pack, or dispenser can be accompanied by instructions for administration.

### EXAMPLES

### Example 1: Study of HIV Vaccine Regimens in Humans

A randomized, double-blind, placebo-controlled, parallel, interventional, Phase 1/2a study in healthy HIV-uninfected adult men and women aged 18 through 50 years is ongoing (this study is referred to as the HPX2004 study herein). A target of 198 subjects participate in this study, randomized into one of 4 subgroups: 55 subjects receive vaccination with Ad26.Mos.HIV and Clade C gp140 in Subgroup 1A, 11 subjects receive placebo in Subgroup 1B, 110 subjects receive vaccination with Ad26.Mos4.HIV and Clade C gp140 in subgroup 2A and 22 subjects receive placebo in Subgroup 2B. Subjects were enrolled regardless of their baseline Ad26 seropositivity. Subjects receive study vaccine or placebo according to the time points in Table 1.

TABLE 1
Study Design

| Group | Subgroup | N | Week 0 | Week 12 | Week 24 | Week 48 |
|---|---|---|---|---|---|---|
| Group 1 | A | 55 | Ad26.Mos.HIV | Ad26.Mos.HIV | Ad26.Mos.HIV + | Ad26.Mos.HIV + |
| | | | | | Clade C gp140 | Clade C gp140 |
| | | | | | (250 mcg + | (250 mcg + |
| | | | | | adjuvant)^{a} | adjuvant)^{a} |
| | B | 11 | Placebo | Placebo | Placebo + | Placebo + |
| | | | | | Placebo | Placebo |
| Group 2 | A | 110 | Ad26.Mos4.HIV | Ad26.Mos4.HIV | Ad26.Mos4.HIV + | Ad26.Mos4.HIV + |
| | | | | | Clade C gp140 | Clade C gp140 |
| | | | | | (250 mcg + | (250 mcg + |
| | | | | | adjuvant)^{a} | adjuvant)^{a} |
| | B | 22 | Placebo | Placebo | Placebo + | Placebo + |
| | | | | | Placebo | Placebo |

^{a}250 mcg refers to total protein content; sterile aluminum phosphate suspension is used as adjuvant. Aluminum content is 0.425 mg/0.5 mL dose.

### Study Vaccines

Ad26.Mos.HIV was composed of the following three vaccine products supplied in the same vial and administered in a 1:1:2 ratio: Ad26.Mos1.Gag-Pol (Ad26 vector encoding a mosaic Gag-Pol fusion protein having SEQ ID NO: 1), Ad26.Mos2.Gag-Pol (Ad26 vector encoding a mosaic Gag-Pol fusion protein having SEQ ID NO: 2), and Ad26.Mos1.Env (Ad26 vector encoding a mosaic Env protein having SEQ ID NO: 3). Total dose per administration is 5×10¹⁰ viral particles (vp) by 0.5 mL intramuscular injection into the deltoid.

Ad26.Mos4.HIV was composed of the following four vaccine products supplied in the same vial and administered in a 1:1:1:1 ratio: Ad26.Mos1.Gag-Pol (Ad26 vector encoding a mosaic Gag-Pol fusion protein having SEQ ID NO: 1), Ad26.Mos2.Gag-Pol (Ad26 vector encoding a mosaic Gag-Pol fusion protein having SEQ ID NO: 2), Ad26.Mos1.Env (Ad26 vector encoding a mosaic Env protein having SEQ ID NO: 3), and Ad26.Mos2S.Env (Ad26 vector encoding a mosaic Env protein having SEQ ID NO: 4). Total dose per administration is 5×10¹⁰ viral particles (vp) by 0.5 mL intramuscular injection into the deltoid.

Clade C gp140 was composed of purified gp140 protein having SEQ ID NO: 5. This was mixed with aluminium phosphate adjuvant at the pharmacy. Dose per administration is 250 microgram total glycoprotein (corresponding to 158 microgram protein, which is another way of indicating the same amount of the Clade C gp140, but has the advantage that it is more consistent in case of potential variability of glycosylation; the absorbtivity constants to calculate the concentrations of HIV Clade C gp140 are 1.25 (mg/mL)⁻¹ cm⁻¹ for glycoprotein and 1.99 (mg/mL)⁻¹ cm⁻¹ for protein, respectively) and 0.425 mg aluminium, per 0.5 mL intramuscular injection into the deltoid.

Placebo was 0.9% saline (0.5 mL injection).

### Safety Evaluations

All adverse events (AEs) and situations requiring special notification are reported from the time a signed and dated informed consent form (ICF) is obtained until 28 days after first dose of study vaccine, and thereafter, pre-dose and for 28 days after each subsequent dose of study vaccine/placebo. All serious AEs (SAES) and AEs leading to discontinuation from the study vaccination (regardless of the causal relationship) and AEs of special interest (AESIs, i.e., confirmed HIV infection) are reported for the duration of the study.

After each vaccination, subjects remain under observation at the study site for at least 30 minutes for presence of any acute reactions and solicited events.

In addition, symptoms of the following solicited AEs are collected via a diary for 7 days post-vaccination (day of vaccination and the subsequent 7 days). The diary is used as a source document.

Solicited local AEs: pain/tenderness, erythema, and swelling/induration (measured using the ruler supplied).

Solicited systemic AEs: fever (temperature measurement), fatigue, headache, nausea, myalgia, and chills.

Temperature is to be measured at approximately the same time each day using the thermometer supplied.

### Immunogenicity Evaluations

Humoral immune response assays include, but are not limited to Env-Ab-binding assays, virus neutralization assay, and assays for Ab functionality.

Cellular immune response assays include, but are not limited to interferon gamma enzyme-linked immunospot assay, intracellular cytokine staining, and multiparameter flow cytometry.

### Objectives

Primary objectives of this study are to assess safety/tolerability of the two different vaccine regimens, and to assess envelope (Env)-binding antibody (Ab) responses of the two different vaccine regimens.

Secondary objectives are to assess neutralizing Ab (nAb) responses, Ab functionality (as assessed by phagocytosis) and Ab isotyping, and to assess T-cell responses.

### Primary Endpoints

AEs throughout the study
   Local and systemic solicited adverse events (AEs) for 7 days post-vaccination.
   AEs for 28 days after each vaccination.
   Discontinuations from vaccination/from study due to AEs.
   Serious AEs (SAES) and AEs of special interest (AESIs) during the course of the study.
Env-specific binding Abs (titers and breadth).

### Secondary Endpoints

Env-specific nAbs (titers and breadth) (for Tier 1 and Tier 2 viruses; note: Tier 2 is assessed only if Tier 1 shows positive results).
Env-specific functional Abs (phagocytosis score and breadth).
Env-specific binding Ab isotypes (IgA, IgG1-4) (titers and breadth).
Interferon (IFN)γ PBMC responders to mosaic peptide pools of Env/group-specific antigen (Gag)/polymerase (Pol) and potential T-cell epitopes (PTE).
Cluster of differentiation (CD)4⁺ and CD8⁺ T-cell functionality (% cells producing Ia, IFNγ, IL-2, IL-4, TNFα).
T-cell development with emphasis on follicular helper T-cells and memory differentiation.
Available samples from time points after last vaccination are used for determination of durability.

### Exploratory Endpoints

Ab functionality evaluation (by other than phagocytosis).
Ab Fc (sub)typing.
Epitope mapping of Ab to Env and T-cell responses to Gag/Pol/Env and PTE.
Regulation of genes (clusters) that predict specific immune responses and human leukocyte antigen typing.
Ab-producing B-cells and characterization of B-cell memory development.
Adenovirus serotype 26 (Ad26) nAbs (titer).

### Safety Results

The vaccines were well tolerated. Adverse events that would have caused the study to have paused, were not reported to date. No SAEs related to vaccines have been reported until the week 28 results. The Week 52 (i.e., 4 weeks post 4^{th} vaccination) analysis results were consistent with the Week 28 (i.e., 4 weeks post 3^{rd} vaccination) primary analysis results and did not reveal any new safety concerns. Both vaccine regimens were found to be well tolerated. The most frequently reported solicited AEs were injection site pain/tenderness, fatigue, headache, and myalgia. One possibly related SAE (suspected unexpected serious adverse reaction, SUSAR) was reported (seronegative rheumatoid arthritis). This isolated report of rheumatoid arthritis did not change the benefit-risk balance for participants enrolled in these adenovector-based clinical vaccine studies. During the vaccination period, no on study HIV infections occurred. Three participants discontinued the study vaccines due to an AE: one participant in the placebo group (urticaria, Grade 1, related to study vaccination), one participant in the placebo group (viral infection, Grade 2, not related to study vaccination), and one participant in the trivalent group (hepatitis C, Grade 1, not related to study vaccination and is still ongoing).

### Immunogenicity Results

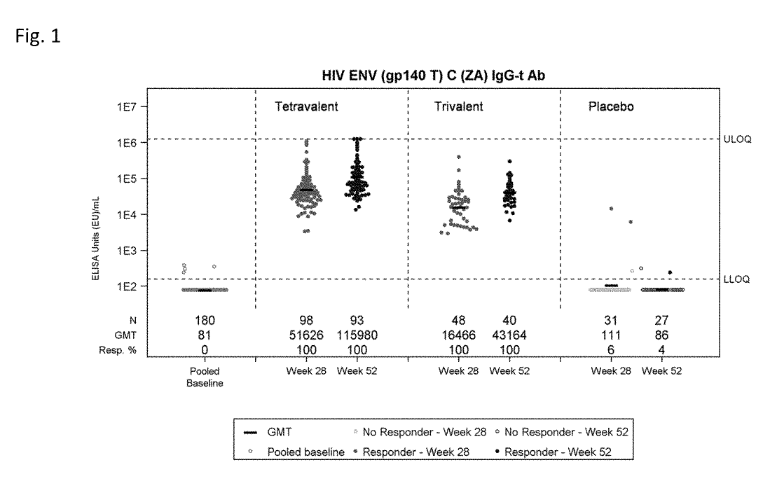

Interim immunogenicity analysis of the HPX2004 study was performed after all participants completed the third and fourth vaccination or discontinued earlier. All active vaccine regimens were immunogenic and induced humoral responses (FIG. 1). To test the vaccine taken after priming, the humoral responses generated by the group of participants who received trivalent Ad26.Mos.HIV group were compared to the tetravalent Ad26.Mos4.HIV group by ELISA at week 16. The responses in the trivalent group were comparable to those seen in HIV-V-A004, a previous study testing this same vaccination, demonstrating the reproducibility of the vaccine regimen between studies. 100% of participants had a detectable humoral response irrespective of vector, but the geometric mean response in the tetravalent group was 1.91-fold higher than the trivalent group.

Four weeks after the third and fourth vaccinations, both groups continued to show 100% of participants having a detectable antibody response. Breadth of responses is also seen, with 100% antibody response rates to diverse clade A, B, C, Consensus C, and Mosaic Env gp140 proteins observed. The fold differences (Geometric Mean Ratio [GMR] and its 95% Confidence Interval) for the ELISA assays after the third and after the fourth vaccinations demonstrated that the addition of the Ad26.Mos2S.Env increased the magnitude of humoral responses not only to autologous antigens but also to Env strains from clades not included in the vaccine, see e.g. Table 2. The difference between the trivalent and tetravalent groups was statistically significant for all 5 antigens tested at both Week 28 and Week 52 at a α=0.05 with P-values based on a 2-sample t-test between the Tetravalent and the Trivalent groups. No antagonism of responses to the Clade B responses, most closely matched to the Ad26.Mos1.Env was seen, which took away a potential concern on the possibility of such interference in humans, and makes the tetravalent Ad26 preferable to trivalent Ad26 for use in all geographic regions, irrespective of the local HIV clades circulating.

TABLE 2
Geometric mean ratios (GMR) upper (UCL) and lower
(LCL) 95% confidence intervals of the tetravalent
Ad26.Mos4.HIV relative to the trivalent Ad26.Mos.HIV
four weeks after the third (Week 28) and 4 weeks after
the fourth (Week 52) vaccinations.

| | Week 28 | | | Week 52 | | |
|---|---|---|---|---|---|---|
| | | 95% CI | | | 95% CI | |
| TEST | GMR | LCL | UCL | GMR | LCL | UCL |
| HIV ENV (gp140 T) A | 2.5 | 1.7 | 3.5 | 3.0 | 2.1 | 4.2 |
| (92UG037.1) IgG-t Ab | | | | | | |
| HIV ENV (gp140 T) B | 1.4 | 1.0 | 1.9 | 1.5 | 1.1 | 2.1 |
| (1990a) IgG-t Ab | | | | | | |
| HIV ENV (gp140 T) C | 2.9 | 2.0 | 4.1 | 1.9 | 1.5 | 2.4 |
| (ConC) IgG-t Ab | | | | | | |
| HIV ENV (gp140 T) C | 3.1 | 2.1 | 4.6 | 2.7 | 1.9 | 3.9 |
| (ZA) IgG-t Ab | | | | | | |
| HIV ENV (gp140 T) | 1.6 | 1.2 | 2.1 | 1.7 | 1.2 | 2.4 |
| Mos1 IgG-t Ab | | | | | | |

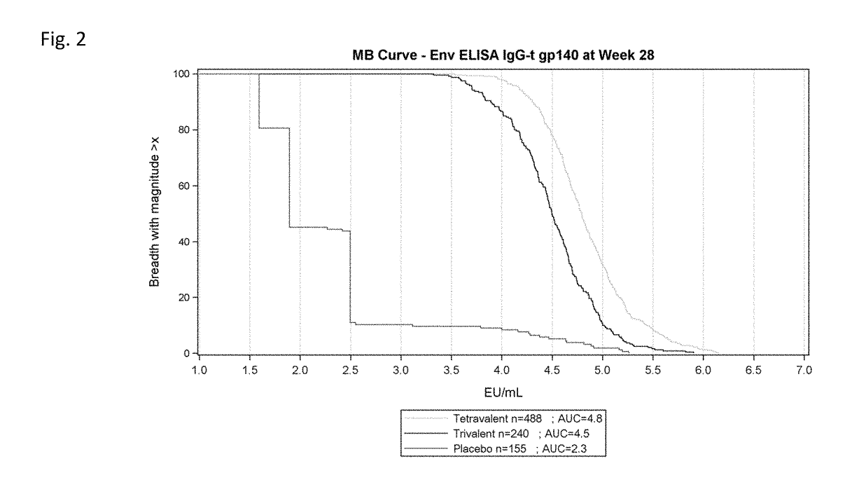

It was also observed that there is an expansion of the area under the magnitude-breadth curve (FIGS. 2 & 3). This indicates that the proportion of study participants with a given antibody response level to any of the 5 gp140 antigens tested is higher for the 4-valent relative to the 3-valent arm. The area under the magnitude-breadth curve (AUC) is substantially higher: 10\hat{ }5.0 for the Ad26.Mos4.HIV relative to the 10\hat{ }4.7 for the Ad26.Mos.HIV. The AUC is a general approximation for the collective humoral response to the panel of antigens tested.

All in all, the data show that the vaccine regimen including Ad26.Mos2S.Env, leads to increased humoral immune responses to HIV envelope glycoprotein in humans as compared to the same regimen without this vector throughout the complete vaccination series.

Sequences
Mos1.GagPol
(SEQ ID NO: 1)
MGARASVLSGGELDRWEKIRLRPGGKKKYRLKHIVWASRELERFAVNPGLLETSEGCRQILGQL
QPSLQTGSEELRSLYNTVATLYCVHQRIEIKDTKEALEKIEEEQNKSKKKAQQAAADTGNSSQV
SQNYPIVQNIQGQMVHQAISPRTLNAWVKVVEEKAFSPEVIPMFSALSEGATPQDLNTMLNTVG
GHQAAMQMLKETINEEAAEWDRVHPVHAGPIAPGQMREPRGSDIAGTTSTLQEQIGWMTNNPPI
PVGEIYKRWIILGLNKIVRMYSPVSILDIRQGPKEPFRDYVDRFYKTLRAEQASQDVKNWMTET
LLVQNANPDCKTILKALGPAATLEEMMTACQGVGGPGHKARVLAEAMSQVTNSATIMMQRGNFR
NQRKTVKCFNCGKEGHIAKNCRAPRKKGCWKCGKEGHQMKDCTERQANFLGKIWPSNKGRPGNF
LQNRPEPTAPPEESFRFGEETTTPSQKQEPIDKEMYPLASLKSLFGNDPSSQMAPISPIETVPV
KLKPGMDGPRVKQWPLTEEKIKALTAICEEMEKEGKITKIGPENPYNTPVFAIKKKDSTKWRKL
VDFRELNKRTQDFWEVQLGIPHPAGLKKKKSVTVLAVGDAYFSVPLDEGFRKYTAFTIPSTNNE
TPGIRYQYNVLPQGWKGSPAIFQCSMTRILEPFRAKNPEIVIYQYMAALYVGSDLEIGQHRAKI
EELREHLLKWGFTTPDKKHQKEPPFLWMGYELHPDKWTVQPIQLPEKDSWTVNDIQKLVGKLNW
ASQIYPGIKVRQLCKLLRGAKALTDIVPLTEEAELELAENREILKEPVHGVYYDPSKDLIAEIQ
KQGHDQWTYQIYQEPFKNLKTGKYAKMRTAHTNDVKQLTEAVQKIAMESIVIWGKTPKFRLPIQ
KETWETWWTDYWQATWIPEWEFVNTPPLVKLWYQLEKDPIAGVETFYVAGAANRETKLGKAGYV
TDRGRQKIVSLTETTNQKTALQAIYLALQDSGSEVNIVTASQYALGIIQAQPDKSESELVNQII
EQLIKKERVYLSWVPAHKGIGGNEQVDKLVSSGIRKVLFLDGIDKAQEEHEKYHSNWRAMASDF
NLPPVVAKEIVASCDQCQLKGEAMHGQVDCSPGIWQLACTHLEGKIILVAVHVASGYIEAEVIP
AETGQETAYFILKLAGRWPVKVIHTANGSNFTSAAVKAACWWAGIQQEFGIPYNPQSQGVVASM
NKELKKIIGQVRDQAEHLKTAVQMAVFIHNFKRKGGIGGYSAGERIIDIIATDIQTKELQKQII
KIQNFRVYYRDSRDPIWKGPAKLLWKGEGAVVIQDNSDIKVVPRRKVKIIKDYGKQMAGADCVA
GRQDED
Mos2.GagPol
(SEQ ID NO: 2)
MGARASILRGGKLDKWEKIRLRPGGKKHYMLKHLVWASRELERFALNPGLLETSEGCKQIIKQL
QPALQTGTEELRSLFNTVATLYCVHAEIEVRDTKEALDKIEEEQNKSQQKTQQAKEADGKVSQN
YPIVQNLQGQMVHQPISPRTLNAWVKVIEEKAFSPEVIPMFTALSEGATPQDLNTMLNTVGGHQ
AAMQMLKDTINEEAAEWDRLHPVHAGPVAPGQMREPRGSDIAGTTSNLQEQIAWMTSNPPIPVG
DIYKRWIILGLNKIVRMYSPTSILDIKQGPKEPFRDYVDRFFKTLRAEQATQDVKNWMTDTLLV
QNANPDCKTILRALGPGATLEEMMTACQGVGGPSHKARVLAEAMSQTNSTILMQRSNFKGSKRI
VKCFNCGKEGHIARNCRAPRKKGCWKCGKEGHQMKDCTERQANFLGKIWPSHKGRPGNFLQSRP
EPTAPPAESFRFEETTPAPKQEPKDREPLTSLRSLFGSDPLSQMAPISPIETVPVKLKPGMDGP
KVKQWPLTEEKIKALVEICTEMEKEGKISKIGPENPYNTPIFAIKKKDSTKWRKLVDFRELNKR
TQDFWEVQLGIPHPAGLKKKKSVTVLAVGDAYFSVPLDEDFRKYTAFTIPSINNETPGIRYQYN
VLPQGWKGSPAIFQSSMTKILEPFRKQNPDIVIYQYMAALYVGSDLEIGQHRTKIEELRQHLLR
WGFTTPDKKHQKEPPFLWMGYELHPDKWTVQPIVLPEKDSWTVNDIQKLVGKLNWASQIYAGIK
VKQLCKLLRGTKALTEVVPLTEEAELELAENREILKEPVHGVYYDPSKDLIAEIQKQGQGQWTY
QIYQEPFKNLKTGKYARMRGAHTNDVKQLTEAVQKIATESIVIWGKTPKFKLPIQKETWEAWWT
EYWQATWIPEWEFVNTPPLVKLWYQLEKEPIVGAETFYVAGAANRETKLGKAGYVTDRGRQKVV
SLTDTTNQKTALQAIHLALQDSGLEVNIVTASQYALGIIQAQPDKSESELVSQIIEQLIKKEKV
YLAWVPAHKGIGGNEQVDKLVSRGIRKVLFLDGIDKAQEEHEKYHSNWRAMASEFNLPPIVAKE
IVASCDKCQLKGEAIHGQVDCSPGIWQLACTHLEGKVILVAVHVASGYIEAEVIPAETGQETAY
FLLKLAGRWPVKTIHTANGSNFTSATVKAACWWAGIKQEFGIPYNPQSQGVVASINKELKKIIG
QVRDQAEHLKTAVQMAVFIHNFKRKGGIGEYSAGERIVDIIASDIQTKELQKQITKIQNFRVYY
RDSRDPLWKGPAKLLWKGEGAVVIQDNSDIKVVPRRKAKIIRDYGKQMAGDDCVASRQDED
Mos1.Env
(SEQ ID NO: 3)
MRVTGIRKNYQHLWRWGTMLLGILMICSAAGKLWVTVYYGVPVWKEATTTLFCASDAKAYDTEV
HNVWATHACVPTDPNPQEVVLENVTENFNMWKNNMVEQMHEDIISLWDQSLKPCVKLTPLCVTL
NCTDDVRNVTNNATNTNSSWGEPMEKGEIKNCSFNITTSIRNKVQKQYALFYKLDVVPIDNDSN
NTNYRLISCNTSVITQACPKVSFEPIPIHYCAPAGFAILKCNDKKFNGTGPCTNVSTVQCTHGI
RPVVSTQLLLNGSLAEEEVVIRSENFTNNAKTIMVQLNVSVEINCTRPNNNTRKSIHIGPGRAF
YTAGDIIGDIRQAHCNISRANWNNTLRQIVEKLGKQFGNNKTIVFNHSSGGDPEIVMHSFNCGG
EFFYCNSTKLFNSTWTWNNSTWNNTKRSNDTEEHITLPCRIKQIINMWQEVGKAMYAPPIRGQI
RCSSNITGLLLTRDGGNDTSGTEIFRPGGGDMRDNWRSELYKYKVVKIEPLGVAPTKAKRRVVQ
SEKSAVGIGAVFLGFLGAAGSTMGAASMTLTVQARLLLSGIVQQQNNLLRAIEAQQHLLQLTVW
GIKQLQARVLAVERYLKDQQLLGIWGCSGKLICTTTVPWNASWSNKSLDKIWNNMTWMEWEREI
NNYTSLIYTLIEESQNQQEKNEQELLELDKWASLWNWFDISNWLW
Mos2S.Env
(SEQ ID NO: 4)
MRVRGMLRNWQQWWIWSSLGFWMLMIYSVMGNLWVTVYYGVPVWKDAKTTLFCASDAKAYEKEV
HNVWATHACVPTDPNPQEIVLGNVTENFNMWKNDMVDQMHEDIISLWDASLEPCVKLTPLCVTL
NCRNVRNVSSNGTYNIIHNETYKEMKNCSFNATTVVEDRKQKVHALFYRLDIVPLDENNSSEKS
SENSSEYYRLINCNTSAITQACPKVSFDPIPIHYCAPAGYAILKCNNKTFNGTGPCNNVSTVQC
THGIKPVVSTQLLLNGSLAEEEIIIRSENLTNNAKTIIVHLNETVNITCTRPNNNTRKSIRIGP
GQTFYATGDIIGDIRQAHCNLSRDGWNKTLQGVKKKLAEHFPNKTIKFAPHSGGDLEITTHTFN
CRGEFFYCNTSNLFNESNIERNDSIITLPCRIKQIINMWQEVGRAIYAPPIAGNITCRSNITGL
LLTRDGGSNNGVPNDTETFRPGGGDMRNNWRSELYKYKVVEVKPLGVAPTEAKRRVVEREKRAV
GIGAVFLGILGAAGSTMGAASITLTVQARQLLSGIVQQQSNLLRAIEAQQHMLQLTVWGIKQLQ
TRVLAIERYLQDQQLLGLWGCSGKLICTTAVPWNTSWSNKSQTDIWDNMTWMQWDKEIGNYTGE
IYRLLEESQNQQEKNEKDLLALDSWNNLWNWFSISKWLWYIKIFIMIVGGLIGLRIIFAVLSIV
NRVRQGY
Clade C gp140 trimeric Env protein
(SEQ ID NO: 5)
AENLWVGNMWVTVYYGVPVWTDAKTTLFCASDTKAYDREVHNVWATHACVPTDPNPQEIVLENV
TENFNMWKNDMVDQMHEDIISLWDQSLKPCVKLTPLCVTLHCTNATFKNNVTNDMNKEIRNCSF
NTTTEIRDKKQQGYALFYRPDIVLLKENRNNSNNSEYILINCNASTITQACPKVNFDPIPIHYC
APAGYAILKCNNKTFSGKGPCNNVSTVQCTHGIKPVVSTQLLLNGSLAEKEIIIRSENLTDNVK
TIIVHLNKSVEIVCTRPNNNTRKSMRIGPGQTFYATGDIIGDIRQAYCNISGSKWNETLKRVKE
KLQENYNNNKTIKFAPSSGGDLEITTHSFNCRGEFFYCNTTRLFNNNATEDETITLPCRIKQII
NMWQGVGRAMYAPPIAGNITCKSNITGLLLVRDGGEDNKTEEIFRPGGGNMKDNWRSELYKYKV
IELKPLGIAPTGAKERVVEREERAVGIGAVFLGFLGAAGSTMGAASLTLTVQARQLLSSIVQQQ
SNLLRAIEAQQHMLQLTVWGIKQLQTRVLAIERYLKDQQLLGIWGCSGKLICTTNVPWNSSWSN
KSQTDIWNNMTWMEWDREISNYTDTIYRLLEDSQTQQEKNEKDLLALDSWKNLWSWFDISNWLW
YIKSRIEGRGSGGYIPEAPRDGQAYVRKDGEWVLLSTFL
Example leader sequence
(SEQ ID NO: 6)
MRVRGIQRNCQHLWRWGTLILGMLMICSA
Mos1.GagPol exemplary nucleotide sequence
(SEQ ID NO: 7)
ATGGGAGCCAGAGCCAGCGTGCTGTCCGGAGGGGAGCTGGACCGCTGGGAGAAGATCAGGCTGAGGCCTG
GAGGGAAGAAGAAGTACAGGCTGAAGCACATCGTGTGGGCCAGCAGAGAGCTGGAACGGTTTGCCGTGAA
CCCTGGCCTGCTGGAAACCAGCGAGGGCTGTAGGCAGATTCTGGGACAGCTGCAGCCCAGCCTGCAGACA
GGCAGCGAGGAACTGCGGAGCCTGTACAACACCGTGGCCACCCTGTACTGCGTGCACCAGCGGATCGAGA
TCAAGGACACCAAAGAAGCCCTGGAAAAGATCGAGGAAGAGCAGAACAAGAGCAAGAAGAAAGCCCAGCA
GGCTGCCGCTGACACAGGCAACAGCAGCCAGGTGTCCCAGAACTACCCCATCGTGCAGAACATCCAGGGA
CAGATGGTGCACCAGGCCATCAGCCCTCGGACCCTGAACGCCTGGGTGAAGGTGGTGGAGGAAAAGGCCT
TCAGCCCTGAGGTGATCCCCATGTTCTCTGCCCTGAGCGAGGGAGCCACACCCCAGGACCTGAACACCAT
GCTGAACACCGTGGGAGGGCACCAGGCTGCCATGCAGATGCTGAAAGAGACAATCAACGAGGAAGCTGCC
GAGTGGGACAGGGTCCACCCAGTGCACGCTGGACCTATCGCTCCTGGCCAGATGAGAGAGCCCAGAGGCA
GCGATATTGCTGGCACCACCTCCACACTGCAGGAACAGATCGGCTGGATGACCAACAACCCTCCCATCCC
TGTGGGAGAGATCTACAAGCGGTGGATCATTCTGGGACTGAACAAGATCGTGCGGATGTACAGCCCTGTG
AGCATCCTGGACATCAGGCAGGGACCCAAAGAGCCCTTCAGGGACTACGTGGACCGGTTCTACAAGACCC
TGAGAGCCGAGCAGGCCAGCCAGGACGTGAAGAACTGGATGACCGAGACACTGCTGGTGCAGAACGCCAA
CCCTGACTGCAAGACCATCCTGAAAGCCCTGGGACCTGCTGCCACCCTGGAAGAGATGATGACAGCCTGC
CAGGGAGTGGGAGGACCTGGCCACAAGGCCAGGGTGCTGGCCGAGGCCATGAGCCAGGTGACCAACTCTG
CCACCATCATGATGCAGAGAGGCAACTTCCGGAACCAGAGAAAGACCGTGAAGTGCTTCAACTGTGGCAA
AGAGGGACACATTGCCAAGAACTGCAGGGCTCCCAGGAAGAAAGGCTGCTGGAAGTGCGGAAAAGAAGGC
CACCAGATGAAGGACTGCACCGAGAGGCAGGCCAACTTCCTGGGCAAGATCTGGCCTAGCAACAAGGGCA
GGCCTGGCAACTTCCTGCAGAACAGACCCGAGCCCACCGCTCCTCCCGAGGAAAGCTTCCGGTTTGGCGA
GGAAACCACCACCCCTAGCCAGAAGCAGGAACCCATCGACAAAGAGATGTACCCTCTGGCCAGCCTGAAG
AGCCTGTTCGGCAACGACCCCAGCAGCCAGATGGCTCCCATCAGCCCAATCGAGACAGTGCCTGTGAAGC
TGAAGCCTGGCATGGACGGACCCAGGGTGAAGCAGTGGCCTCTGACCGAGGAAAAGATCAAAGCCCTGAC
AGCCATCTGCGAGGAAATGGAAAAAGAGGGCAAGATCACCAAGATCGGACCCGAGAACCCCTACAACACC
CCTGTGTTCGCCATCAAGAAGAAAGACAGCACCAAGTGGAGGAAACTGGTGGACTTCAGAGAGCTGAACA
AGCGGACCCAGGACTTCTGGGAGGTGCAGCTGGGCATCCCTCACCCTGCTGGCCTGAAGAAAAAGAAAAG
CGTGACCGTGCTGGCTGTGGGAGATGCCTACTTCAGCGTGCCTCTGGACGAGGGCTTCCGGAAGTACACA
GCCTTCACCATCCCCAGCACCAACAACGAGACACCTGGCATCAGATACCAGTACAACGTGCTGCCTCAGG
GCTGGAAAGGCAGCCCTGCCATCTTCCAGTGCAGCATGACCAGAATCCTGGAACCCTTCAGAGCCAAGAA
CCCTGAGATCGTGATCTACCAGTATATGGCTGCCCTCTACGTGGGCAGCGACCTGGAAATCGGACAGCAC
AGAGCCAAAATCGAAGAACTCCGCGAGCACCTGCTGAAGTGGGGATTCACCACCCCTGACAAGAAGCACC
AGAAAGAGCCTCCCTTCCTGTGGATGGGCTACGAGCTGCACCCTGACAAGTGGACCGTGCAGCCCATCCA
GCTGCCAGAGAAGGACTCCTGGACCGTGAACGACATCCAGAAACTGGTCGGCAAGCTGAACTGGGCCAGC
CAGATCTACCCTGGCATCAAAGTCAGACAGCTGTGTAAGCTGCTGAGGGGAGCCAAAGCACTGACCGACA
TCGTGCCTCTGACAGAAGAAGCCGAGCTGGAACTGGCCGAGAACAGAGAGATCCTGAAAGAACCCGTGCA
CGGAGTGTACTACGACCCCTCCAAGGACCTGATTGCCGAGATCCAGAAACAGGGACACGACCAGTGGACC
TACCAGATCTATCAGGAACCTTTCAAGAACCTGAAAACAGGCAAGTACGCCAAGATGCGGACAGCCCACA
CCAACGACGTGAAGCAGCTGACCGAAGCCGTGCAGAAAATCGCCATGGAAAGCATCGTGATCTGGGGAAA
GACACCCAAGTTCAGGCTGCCCATCCAGAAAGAGACATGGGAAACCTGGTGGACCGACTACTGGCAGGCC
ACCTGGATTCCCGAGTGGGAGTTCGTGAACACCCCACCCCTGGTGAAGCTGTGGTATCAGCTGGAAAAGG
ACCCTATCGCTGGCGTGGAGACATTCTACGTGGCTGGAGCTGCCAACAGAGAGACAAAGCTGGGCAAGGC
TGGCTACGTGACCGACAGAGGCAGACAGAAAATCGTGAGCCTGACCGAAACCACCAACCAGAAAACAGCC
CTGCAGGCCATCTATCTGGCACTGCAGGACAGCGGAAGCGAGGTGAACATCGTGACAGCCAGCCAGTATG
CCCTGGGCATCATCCAGGCCCAGCCTGACAAGAGCGAGAGCGAGCTGGTGAACCAGATCATCGAGCAGCT
GATCAAGAAAGAACGGGTGTACCTGAGCTGGGTGCCAGCCCACAAGGGCATCGGAGGGAACGAGCAGGTG
GACAAGCTGGTGTCCAGCGGAATCCGGAAGGTGCTGTTCCTGGACGGCATCGATAAAGCCCAGGAAGAGC
ACGAGAAGTACCACAGCAATTGGAGAGCCATGGCCAGCGACTTCAACCTGCCTCCCGTGGTGGCCAAAGA
AATCGTGGCCAGCTGCGACCAGTGCCAGCTGAAAGGCGAGGCCATGCACGGACAGGTGGACTGCTCCCCT
GGCATCTGGCAGCTGGCATGCACCCACCTGGAAGGCAAGATCATTCTGGTGGCCGTGCACGTGGCCAGCG
GATACATCGAAGCCGAAGTGATCCCTGCCGAGACAGGGCAGGAAACAGCCTACTTCATCCTGAAGCTGGC
TGGCAGATGGCCTGTGAAGGTGATCCACACAGCCAACGGCAGCAACTTCACCTCTGCTGCCGTGAAGGCT
GCCTGTTGGTGGGCTGGCATTCAGCAGGAATTTGGCATCCCCTACAATCCCCAGTCTCAGGGAGTGGTGG
CCAGCATGAACAAAGAGCTGAAGAAGATCATCGGACAGGTCAGGGATCAGGCCGAGCACCTGAAAACTGC
CGTCCAGATGGCCGTGTTCATCCACAACTTCAAGCGGAAGGGAGGGATCGGAGGGTACTCTGCTGGCGAG
CGGATCATCGACATCATTGCCACCGATATCCAGACCAAAGAGCTGCAGAAACAGATCATCAAGATCCAGA
ACTTCAGGGTGTACTACAGGGACAGCAGGGACCCCATCTGGAAGGGACCTGCCAAGCTGCTGTGGAAAGG
CGAAGGAGCCGTCGTCATCCAGGACAACAGCGACATCAAGGTGGTGCCCAGACGGAAGGTGAAAATCATC
AAGGACTACGGCAAACAGATGGCTGGAGCCGACTGTGTCGCTGGCAGGCAGGACGAGGACTAATGA
Mos2.GagPol exemplary nucleotide sequence
(SEQ ID NO: 8)
ATGGGAGCCAGAGCCAGCATCCTGCGAGGAGGGAAGCTGGACAAGTGGGAGAAGATCAGGCTGAGGCCTG
GAGGGAAGAAACACTACATGCTGAAGCACCTGGTCTGGGCCAGCAGAGAGCTGGAACGGTTTGCCCTCAA
TCCTGGCCTGCTGGAAACCAGCGAGGGCTGCAAGCAGATCATCAAGCAGCTGCAGCCTGCCCTGCAGACA
GGCACCGAGGAACTGCGGAGCCTGTTCAACACCGTGGCCACCCTGTACTGCGTGCATGCCGAGATCGAAG
TGAGGGACACCAAAGAAGCCCTGGACAAGATCGAGGAAGAGCAGAACAAGAGCCAGCAGAAAACCCAGCA
GGCCAAAGAAGCCGACGGCAAGGTCTCCCAGAACTACCCCATCGTGCAGAACCTGCAGGGACAGATGGTG
CACCAGCCCATCAGCCCTCGGACACTGAATGCCTGGGTGAAGGTGATCGAGGAAAAGGCCTTCAGCCCTG
AGGTGATCCCCATGTTCACAGCCCTGAGCGAGGGAGCCACACCCCAGGACCTGAACACCATGCTGAACAC
CGTGGGAGGGCACCAGGCTGCCATGCAGATGCTGAAGGACACCATCAACGAGGAAGCTGCCGAGTGGGAC
AGGCTGCACCCTGTGCACGCTGGACCTGTGGCTCCTGGCCAGATGAGAGAGCCCAGAGGCAGCGATATTG
CTGGCACCACCTCCAATCTGCAGGAACAGATCGCCTGGATGACCAGCAACCCTCCCATCCCTGTGGGAGA
CATCTACAAGCGGTGGATCATCCTGGGACTGAACAAGATCGTGCGGATGTACAGCCCTACCTCCATCCTG
GACATCAAGCAGGGACCCAAAGAGCCTTTCAGGGACTACGTGGACCGGTTCTTCAAGACCCTGAGAGCCG
AGCAGGCCACCCAGGACGTGAAGAACTGGATGACCGACACCCTGCTGGTGCAGAACGCCAACCCTGACTG
CAAGACCATCCTGAGAGCCCTGGGACCTGGAGCCACCCTGGAAGAGATGATGACAGCCTGCCAGGGAGTG
GGAGGACCCTCTCACAAGGCTAGGGTGCTGGCCGAGGCCATGAGCCAGACCAACAGCACCATCCTGATGC
AGCGGAGCAACTTCAAGGGCAGCAAGCGGATCGTGAAGTGCTTCAACTGTGGCAAAGAGGGACACATTGC
CAGAAACTGTAGGGCACCCAGGAAGAAAGGCTGCTGGAAGTGCGGAAAAGAAGGCCACCAGATGAAGGAC
TGCACCGAGAGGCAGGCCAACTTCCTGGGCAAGATCTGGCCTAGCCACAAGGGCAGACCTGGCAACTTCC
TGCAGAGCAGACCCGAGCCCACCGCTCCTCCAGCCGAGAGCTTCCGGTTCGAGGAAACCACCCCTGCTCC
CAAGCAGGAACCTAAGGACAGAGAGCCTCTGACCAGCCTGAGAAGCCTGTTCGGCAGCGACCCTCTGAGC
CAGATGGCTCCCATCTCCCCTATCGAGACAGTGCCTGTGAAGCTGAAGCCTGGCATGGACGGACCCAAGG
TGAAACAGTGGCCTCTGACCGAGGAAAAGATCAAAGCCCTGGTGGAGATCTGTACCGAGATGGAAAAAGA
GGGCAAGATCAGCAAGATCGGACCCGAGAACCCCTACAACACCCCTATCTTCGCCATCAAGAAGAAAGAC
AGCACCAAGTGGAGGAAACTGGTGGACTTCAGAGAGCTGAACAAGCGGACCCAGGACTTCTGGGAGGTGC
AGCTGGGCATCCCTCACCCTGCTGGCCTGAAGAAAAAGAAAAGCGTGACCGTGCTGGCCGTGGGAGATGC
CTACTTCAGCGTGCCTCTGGACGAGGACTTCAGAAAGTACACAGCCTTCACCATCCCCAGCATCAACAAC
GAGACACCTGGCATCAGATACCAGTACAACGTGCTGCCTCAGGGATGGAAGGGCTCTCCTGCAATCTTCC
AGAGCAGCATGACCAAGATCCTGGAACCCTTCCGGAAGCAGAACCCTGACATCGTGATCTACCAGTACAT
GGCAGCCCTGTACGTCGGCAGCGACCTGGAAATCGGACAGCACCGGACCAAGATCGAAGAACTCAGGCAG
CACCTGCTGCGGTGGGGATTCACCACCCCTGACAAGAAGCACCAGAAAGAGCCTCCCTTCCTGTGGATGG
GCTACGAGCTGCACCCAGACAAGTGGACCGTGCAGCCCATCGTGCTGCCTGAGAAGGACTCCTGGACCGT
GAACGACATCCAGAAACTGGTCGGCAAGCTGAACTGGGCCAGCCAGATCTACGCTGGCATCAAAGTGAAG
CAGCTGTGTAAGCTCCTGAGAGGCACCAAAGCCCTGACCGAGGTGGTGCCACTGACAGAGGAAGCCGAGC
TGGAACTGGCCGAGAACAGAGAGATCCTGAAAGAACCCGTGCACGGAGTGTACTACGACCCCAGCAAGGA
CCTGATTGCCGAGATCCAGAAGCAGGGACAGGGACAGTGGACCTACCAGATCTACCAGGAACCCTTCAAG
AACCTGAAAACAGGCAAGTACGCCAGGATGAGGGGAGCCCACACCAACGACGTCAAACAGCTGACCGAAG
CCGTGCAGAAGATCGCCACCGAGAGCATCGTGATTTGGGGAAAGACACCCAAGTTCAAGCTGCCCATCCA
GAAAGAGACATGGGAGGCCTGGTGGACCGAGTACTGGCAGGCCACCTGGATTCCCGAGTGGGAGTTCGTG
AACACCCCACCCCTGGTGAAGCTGTGGTATCAGCTGGAAAAAGAACCCATCGTGGGAGCCGAGACATTCT
ACGTGGCTGGAGCTGCCAACAGAGAGACAAAGCTGGGCAAGGCTGGCTACGTGACCGACAGAGGCAGGCA
GAAAGTGGTGTCCCTGACCGATACCACCAACCAGAAAACAGCCCTGCAGGCCATCCACCTGGCTCTGCAG
GACTCTGGCCTGGAAGTGAACATCGTGACAGCCAGCCAGTATGCCCTGGGCATCATTCAGGCACAGCCTG
ACAAGAGCGAGAGCGAGCTGGTGTCTCAGATCATTGAGCAGCTGATCAAGAAAGAAAAGGTGTACCTGGC
CTGGGTGCCAGCCCACAAGGGGATCGGAGGGAACGAGCAGGTGGACAAGCTGGTGTCCAGGGGCATCCGG
AAGGTGCTGTTTCTGGACGGCATCGACAAAGCCCAGGAAGAGCACGAGAAGTACCACAGCAATTGGAGAG
CCATGGCCAGCGAGTTCAACCTGCCTCCCATCGTGGCCAAAGAAATCGTGGCCTCTTGCGACAAGTGCCA
GCTGAAAGGCGAGGCCATTCACGGACAGGTGGACTGCAGCCCAGGCATCTGGCAGCTGGCCTGCACCCAC
CTGGAAGGCAAGGTGATCCTGGTGGCCGTGCACGTGGCCTCTGGATACATCGAAGCCGAAGTGATCCCTG
CCGAGACAGGCCAGGAAACAGCCTACTTCCTGCTGAAGCTGGCTGGCAGGTGGCCTGTGAAAACCATCCA
CACAGCCAACGGCAGCAACTTCACCTCTGCCACCGTGAAGGCTGCCTGTTGGTGGGCTGGCATTAAGCAG
GAATTTGGCATCCCCTACAACCCTCAGTCTCAGGGAGTGGTGGCCTCCATCAACAAAGAGCTGAAGAAGA
TCATCGGACAGGTCAGGGATCAGGCCGAGCATCTGAAAACAGCCGTCCAGATGGCCGTGTTCATCCACAA
CTTCAAGCGGAAGGGAGGGATCGGAGAGTACTCTGCTGGCGAGAGGATCGTGGACATTATCGCCAGCGAT
ATCCAGACCAAAGAACTGCAGAAGCAGATCACAAAGATCCAGAACTTCAGGGTGTACTACAGGGACAGCA
GAGATCCCCTGTGGAAGGGACCTGCCAAGCTGCTGTGGAAAGGCGAAGGAGCCGTCGTCATCCAGGACAA
CAGCGACATCAAGGTGGTGCCCAGACGGAAGGCCAAGATCATCAGAGACTACGGCAAACAGATGGCTGGC
GACGACTGCGTCGCCTCTAGGCAGGACGAGGACTAATGA
Mos1.Env exemplary nucleotide sequence
(SEQ ID NO: 9)
ATGCGGGTGACCGGCATCCGGAAGAACTACCAGCACCTGTGGCGGTGGGGCACCATGCTGCTGGGCATCC
TGATGATTTGCTCTGCCGCCGGAAAGCTGTGGGTGACCGTGTACTACGGCGTGCCCGTGTGGAAAGAGGC
CACCACCACCCTGTTCTGCGCCAGCGACGCCAAGGCCTACGACACCGAGGTGCACAACGTGTGGGCCACC
CACGCCTGCGTGCCCACCGACCCCAACCCCCAGGAAGTGGTCCTGGAAAACGTGACCGAGAACTTCAACA
TGTGGAAGAACAACATGGTGGAGCAGATGCACGAGGACATCATCAGCCTGTGGGACCAGAGCCTGAAGCC
CTGCGTGAAGCTGACCCCCCTGTGCGTGACCCTGAACTGCACCGACGACGTGCGGAACGTGACCAACAAC
GCCACCAACACCAACAGCAGCTGGGGCGAGCCTATGGAAAAGGGCGAGATCAAGAACTGCAGCTTCAACA
TCACCACCTCCATCCGGAACAAGGTGCAGAAGCAGTACGCCCTGTTCTACAAGCTGGACGTGGTGCCCAT
CGACAACGACAGCAACAACACCAACTACCGGCTGATCAGCTGCAACACCAGCGTGATCACCCAGGCCTGC
CCCAAGGTGTCCTTCGAGCCCATCCCCATCCACTACTGCGCCCCTGCCGGCTTCGCCATCCTGAAGTGCA
ACGACAAGAAGTTCAACGGCACCGGCCCCTGCACCAACGTGAGCACCGTGCAGTGCACCCACGGCATCCG
GCCCGTGGTGTCCACCCAGCTGCTGCTGAACGGCAGCCTGGCCGAGGAAGAGGTGGTGATCAGAAGCGAG
AATTTCACCAACAATGCCAAGACCATCATGGTGCAGCTGAACGTGAGCGTGGAGATCAACTGCACCCGGC
CCAACAACAACACCCGGAAGAGCATCCACATCGGCCCTGGCAGGGCCTTCTACACAGCCGGCGACATCAT
CGGCGACATCCGGCAGGCCCACTGCAACATCAGCCGGGCCAACTGGAACAACACCCTGCGGCAGATCGTG
GAGAAGCTGGGCAAGCAGTTCGGCAACAACAAGACCATCGTGTTCAACCACAGCAGCGGCGGAGACCCCG
AGATCGTGATGCACAGCTTCAACTGTGGCGGCGAGTTCTTCTACTGCAACAGCACCAAGCTGTTCAACAG
CACCTGGACCTGGAACAACTCCACCTGGAATAACACCAAGCGGAGCAACGACACCGAAGAGCACATCACC
CTGCCCTGCCGGATCAAGCAGATTATCAATATGTGGCAGGAGGTCGGCAAGGCCATGTACGCCCCTCCCA
TCCGGGGCCAGATCCGGTGCAGCAGCAACATCACCGGCCTGCTGCTGACCCGGGACGGCGGCAACGATAC
CAGCGGCACCGAGATCTTCCGGCCTGGCGGCGGAGATATGCGGGACAACTGGCGGAGCGAGCTGTACAAG
TACAAGGTGGTGAAGATCGAGCCCCTGGGCGTGGCTCCCACCAAGGCCAAGCGGCGGGTGGTGCAGAGCG
AGAAGAGCGCCGTGGGCATCGGCGCCGTGTTTCTGGGCTTCCTGGGAGCCGCCGGAAGCACCATGGGAGC
CGCCAGCATGACCCTGACCGTGCAGGCCCGGCTGCTGCTGTCCGGCATCGTGCAGCAGCAGAACAACCTG
CTCCGGGCCATCGAGGCCCAGCAGCACCTGCTGCAGCTGACCGTGTGGGGCATCAAGCAGCTGCAGGCCA
GGGTGCTGGCCGTGGAGAGATACCTGAAGGATCAGCAGCTCCTGGGGATCTGGGGCTGCAGCGGCAAGCT
GATCTGCACCACCACCGTGCCCTGGAACGCCAGCTGGTCCAACAAGAGCCTGGACAAGATCTGGAACAAT
ATGACCTGGATGGAATGGGAGCGCGAGATCAACAATTACACCAGCCTGATCTACACCCTGATCGAGGAAA
GCCAGAACCAGCAGGAAAAGAACGAGCAGGAACTGCTGGAACTGGACAAGTGGGCCAGCCTGTGGAACTG
GTTCGACATCAGCAACTGGCTGTGGTAATGA
Mos2S.Env exemplary nucleotide sequence
(SEQ ID NO: 10)
ATGAGAGTGCGGGGCATGCTGAGAAACTGGCAGCAGTGGTGGATCTGGTCCAGCCTGGGCTTCTGGATGC
TGATGATCTACAGCGTGATGGGCAACCTGTGGGTCACCGTGTACTACGGCGTGCCCGTGTGGAAGGACGC
CAAGACCACCCTGTTTTGCGCCTCCGATGCCAAGGCCTACGAGAAAGAGGTGCACAACGTCTGGGCCACC
CACGCCTGTGTGCCCACCGACCCCAATCCCCAGGAAATCGTCCTGGGCAACGTGACCGAGAACTTCAACA
TGTGGAAGAACGACATGGTCGATCAGATGCACGAGGACATCATCTCCCTGTGGGACGCCTCCCTGGAACC
CTGCGTGAAGCTGACCCCTCTGTGCGTGACCCTGAACTGCCGGAACGTGCGCAACGTGTCCAGCAACGGC
ACCTACAACATCATCCACAACGAGACATACAAAGAGATGAAGAACTGCAGCTTCAACGCTACCACCGTGG
TCGAGGACCGGAAGCAGAAGGTGCACGCCCTGTTCTACCGGCTGGACATCGTGCCCCTGGACGAGAACAA
CAGCAGCGAGAAGTCCTCCGAGAACAGCTCCGAGTACTACAGACTGATCAACTGCAACACCAGCGCCATC
ACCCAGGCCTGCCCCAAGGTGTCCTTCGACCCTATCCCCATCCACTACTGCGCCCCTGCCGGCTACGCCA
TCCTGAAGTGCAACAACAAGACCTTCAATGGCACCGGCCCCTGCAACAATGTGTCCACCGTGCAGTGCAC
CCACGGCATCAAGCCCGTGGTGTCTACCCAGCTGCTGCTGAACGGCAGCCTGGCCGAGGAAGAGATCATT
ATCAGAAGCGAGAACCTGACCAACAACGCCAAAACCATCATCGTCCACCTGAACGAAACCGTGAACATCA
CCTGTACCCGGCCTAACAACAACACCCGGAAGTCCATCCGGATCGGCCCTGGCCAGACCTTTTACGCCAC
CGGCGATATTATCGGCGACATCCGGCAGGCCCACTGCAATCTGAGCCGGGACGGCTGGAACAAGACACTG
CAGGGCGTCAAGAAGAAGCTGGCCGAACACTTCCCTAACAAGACTATCAAGTTCGCCCCTCACTCTGGCG
GCGACCTGGAAATCACCACCCACACCTTCAACTGTCGGGGCGAGTTCTTCTACTGCAATACCTCCAACCT
GTTCAACGAGAGCAACATCGAGCGGAACGACAGCATCATCACACTGCCTTGCCGGATCAAGCAGATTATC
AATATGTGGCAGGAAGTGGGCAGAGCCATCTACGCCCCTCCAATCGCCGGCAACATCACATGCCGGTCCA
ATATCACCGGCCTGCTGCTCACCAGAGATGGCGGCTCCAACAATGGCGTGCCAAACGACACCGAGACATT
CAGACCCGGCGGAGGCGACATGCGGAACAATTGGCGGAGCGAGCTGTACAAGTACAAGGTGGTGGAAGTG
AAGCCCCTGGGCGTGGCCCCTACCGAGGCCAAGAGAAGAGTGGTCGAACGCGAGAAGCGGGCCGTGGGAA
TCGGAGCCGTGTTTCTGGGAATCCTGGGAGCCGCTGGCTCTACCATGGGCGCTGCCTCTATCACCCTGAC
AGTGCAGGCCAGACAGCTGCTCAGCGGCATCGTGCAGCAGCAGAGCAACCTGCTGAGAGCCATTGAGGCC
CAGCAGCACATGCTGCAGCTGACCGTGTGGGGCATTAAGCAGCTCCAGACACGGGTGCTGGCCATCGAGA
GATACCTGCAGGATCAGCAGCTCCTGGGCCTGTGGGGCTGTAGCGGCAAGCTGATCTGTACCACCGCCGT
GCCCTGGAATACCTCTTGGAGCAACAAGAGCCAGACCGACATCTGGGACAACATGACCTGGATGCAGTGG
GACAAAGAAATCGGCAACTATACCGGCGAGATCTATAGACTGCTGGAAGAGTCCCAGAACCAGCAGGAAA
AGAACGAGAAGGACCTGCTGGCCCTGGATTCTTGGAACAATCTGTGGAACTGGTTCAGCATCTCCAAGTG
GCTGTGGTACATCAAGATCTTCATCATGATCGTGGGCGGCCTGATCGGCCTGCGGATCATCTTTGCCGTG
CTGAGCATCGTGAACCGCGTGCGGCAGGGCTACTGATAA
Exemplary nucleic acid sequence encoding Clade C gp140 trimeric
Env protein, including leader sequence
(SEQ ID NO: 11)
ATGAGAGTGCGGGGCATCCAGCGGAACTGCCAGCATCTGTGGCGCTGGGGCACCCTGATCCTGGGCATGC
TGATGATCTGCTCTGCCGCCGAGAACCTGTGGGTCGGAAATATGTGGGTCACCGTGTACTACGGCGTGCC
CGTGTGGACCGACGCCAAGACCACCCTGTTCTGCGCCAGCGACACCAAGGCCTACGACCGCGAGGTGCAC
AACGTGTGGGCCACCCACGCTTGTGTGCCTACCGACCCCAACCCCCAGGAAATCGTCCTGGAAAACGTGA
CCGAGAACTTCAACATGTGGAAGAACGACATGGTGGACCAGATGCACGAGGACATCATCAGCCTGTGGGA
CCAGAGCCTGAAGCCCTGCGTGAAGCTGACACCCCTGTGCGTGACCCTGCACTGCACCAACGCCACCTTC
AAGAACAACGTGACCAACGACATGAACAAAGAGATCCGGAACTGCAGCTTCAACACCACCACCGAGATCC
GGGACAAGAAGCAGCAGGGCTACGCCCTGTTCTACCGGCCCGACATCGTGCTGCTGAAAGAGAACAGAAA
CAACAGCAACAACTCCGAGTACATCCTGATCAACTGCAACGCCAGCACCATCACCCAGGCCTGCCCCAAA
GTGAACTTCGACCCTATCCCCATCCACTACTGCGCCCCTGCCGGCTACGCCATCCTGAAGTGCAACAACA
AGACCTTCAGCGGCAAGGGCCCCTGCAACAACGTGTCCACCGTGCAGTGCACCCACGGCATCAAGCCCGT
GGTGTCCACCCAGCTGCTGCTGAATGGCAGCCTGGCCGAGAAAGAGATCATCATCAGAAGCGAGAACCTG
ACCGACAACGTCAAGACCATCATCGTGCACCTGAACAAGAGCGTGGAAATCGTGTGCACCCGGCCCAACA
ACAACACCAGAAAGAGCATGCGGATCGGCCCTGGCCAGACCTTCTACGCCACCGGCGACATCATCGGCGA
CATCCGGCAGGCCTACTGCAACATCAGCGGCAGCAAGTGGAACGAGACACTGAAGAGAGTGAAAGAGAAG
CTGCAGGAAAACTATAACAACAACAAAACCATCAAGTTCGCCCCCAGCTCTGGCGGCGACCTGGAAATCA
CCACCCACAGCTTCAACTGCAGAGGCGAGTTCTTCTACTGCAATACCACCCGGCTGTTCAACAACAACGC
CACCGAGGACGAGACAATCACCCTGCCCTGCCGGATCAAGCAGATCATCAATATGTGGCAGGGCGTGGGC
AGAGCTATGTACGCCCCTCCTATCGCCGGCAACATCACATGCAAGAGCAACATCACCGGCCTGCTGCTCG
TGCGGGACGGCGGCGAGGATAACAAGACCGAGGAAATCTTCAGACCCGGCGGAGGCAACATGAAGGACAA
CTGGCGGAGCGAGCTGTACAAGTACAAAGTGATCGAGCTGAAGCCCCTGGGAATCGCCCCTACCGGAGCC
AAGGAAAGAGTGGTGGAACGCGAGGAGCGGGCCGTGGGAATCGGCGCCGTGTTCCTGGGCTTTCTGGGAG
CCGCCGGAAGCACAATGGGCGCTGCCAGCCTGACCCTGACCGTGCAGGCCAGACAGCTGCTGAGCAGCAT
CGTGCAGCAGCAGAGCAACCTCCTGAGAGCCATCGAGGCCCAGCAGCACATGCTGCAGCTGACCGTGTGG
GGCATCAAGCAGCTGCAGACCCGGGTGCTGGCCATCGAGAGATACCTGAAGGACCAGCAGCTCCTGGGCA
TCTGGGGCTGCAGCGGCAAGCTGATCTGCACCACCAACGTGCCCTGGAACAGCAGCTGGTCCAACAAGAG
CCAGACCGACATCTGGAACAACATGACCTGGATGGAATGGGACCGCGAGATCAGCAACTACACCGACACC
ATCTACCGGCTGCTGGAAGATAGCCAGACCCAGCAGGAAAAGAACGAGAAGGACCTGCTGGCCCTGGACA
GCTGGAAGAATCTGTGGTCTTGGTTTGACATCAGCAACTGGCTGTGGTACATCAAGAGCCGGATCGAGGG
CAGAGGCAGCGGCGGCTACATCCCTGAGGCCCCTAGAGATGGCCAGGCCTACGTGCGGAAGGACGGCGAA
TGGGTGCTGCTGTCCACCTTCCTGTGATAA

1. A method of inducing a safe immune response against multiple clades of human immunodeficiency virus (HIV) in a human subject in need thereof, comprising:
(1) administering to the subject a priming composition comprising one or more Ad26 vectors together encoding at least the antigenic polypeptides having amino acid sequences that are at least 95% identical to SEQ ID NO: 3 and SEQ ID NO: 4 respectively, and a pharmaceutically acceptable carrier;
(2) administering to the subject, the priming composition;
(3) administering to the subject, a first boosting composition comprising at least one isolated HIV envelope glycoprotein, an adjuvant and a pharmaceutically acceptable carrier; and
(4) administering to the subject, together with (3), a second boosting composition comprising one or more Ad26 vectors together encoding at least the antigenic polypeptides having amino acid sequences that are at least 95% identical to SEQ ID NO: 3 and SEQ ID NO: 4 respectively, and a pharmaceutically acceptable carrier,
wherein the immune response comprises an antibody response that includes IgG that binds to isolated HIV envelope glycoproteins from strains of at least clades A, B, and C, when measured in enzyme-linked immunosorbent assays (ELISAs).

2. The method of claim 1, further comprising after step (4):
(5) administering to the subject, the first boosting composition; and
(6) administering to the subject, together with (5), the second boosting composition.

3. The method of claim 1, wherein the priming composition and second boosting composition further comprise one or more Ad26 vectors together encoding the antigenic polypeptides having amino acid sequences that are at least 95% identical to SEQ ID NO: 1 and SEQ ID NO: 2 respectively.

4. The method of claim 1, wherein the at least one isolated HIV envelope glycoprotein in the first boosting composition has an amino acid sequence that is at least 95% identical to SEQ ID NO: 5.

5. The method of claim 1, wherein the adjuvant in the first boosting composition is aluminium phosphate.

6. The method of claim 1, wherein in each step wherein the Ad26 vectors are administered, these are administered at a total dose of about 5×10⁹ to about 1×10¹¹ vp, and
wherein in each step wherein the isolated HIV envelope glycoprotein is administered, this is administered at a total dose of about 125 μg to 350 μg glycoprotein.

7. The method of claim 1, wherein
step (2) is performed at about 10-14 weeks after step (1),
steps (3) and (4) are performed at about 22-26 weeks after step (1),
and optionally steps (5) and (6) are performed at about 42-60 weeks after step (1).

8. The method of claim 1, wherein the priming composition and the second boosting composition each comprise a first Ad26 vector encoding SEQ ID NO: 1, a second Ad26 vector encoding SEQ ID NO: 2, a third Ad26 vector encoding SEQ ID NO: 3, and a fourth Ad26 vector encoding SEQ ID NO: 4.

9. The method of claim 1, wherein the antibody response has a response rate of at least 90%.

10. The method of claim 1, wherein at least at step (1) the human subject is seronegative for HIV.

11. The method of claim 1, wherein the antibody response is at least 1.5 fold higher in magnitude to isolated HIV envelope glycoprotein of at least a clade C strain, as compared to the same vaccine regimen wherein an Ad26 vector encoding SEQ ID NO: 4 is replaced by an Ad26 vector encoding SEQ ID NO: 3.

12. The method of claim 1, wherein step (2) is performed about 12 weeks after step (1), and steps (3) and (4) are performed about 24 weeks after step (1).

13. The method of claim 2, wherein steps (5) and (6) are performed about 48 weeks after step (1).

14. The method of claim 1, wherein in each step wherein the Ad26 vectors are administered, these are administered at a total dose of about 5×10¹⁰ vp.

15. The method of claim 1, wherein in each step wherein the isolated HIV envelope glycoprotein is administered, this is administered at a dose of about 250 μg glycoprotein.

16. The method of claim 1, wherein the human subject resides in an area or country where the predominant clade for HIV infections in humans is Clade A, Clade B, or Clade C, or a circulating recombinant form (CRF) derived from recombination between different clades of which at least one is Clade A, Clade B, or Clade C.

17. The method of claim 16, wherein the human subject resides in an area or country wherein the predominant clade for HIV infections is Clade C.

18. The method of claim 16, wherein the human subject resides in an area or country wherein the predominant clade for HIV infections is Clade B.

19. The method of claim 1, wherein the priming composition and second boosting composition comprise one or more Ad26 vectors together encoding at least the antigenic polypeptides having the amino acid sequences of SEQ ID NO: 3 and SEQ ID NO: 4.

20. The method of claim 3, wherein the priming composition and second boosting composition further comprise one or more Ad26 vectors together encoding the antigenic polypeptides having the amino acid sequences of SEQ ID NO: 1 and SEQ ID NO: 2.

21. The method of claim 4, wherein the at least one isolated HIV envelope glycoprotein in the first boosting composition has an amino acid sequence of SEQ ID NO: 5.


SEQUENCE LISTING (11 sequences)

SEQ ID NO: 1 - protein, 1350 aa, Artificial Sequence
  Mos1.GagPol
MGARASVLSGGELDRWEKIRLRPGGKKKYRLKHIVWASRELERFAVNPGLLETSEGCRQILGQLQPSLQTGSEELRSLYNTVATLYCVHQRIEIKDTKEALEKIEEEQNKSKKKAQQAAADTGNSSQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPVSILDIRQGPKEPFRDYVDRFYKTLRAEQASQDVKNWMTETLLVQNANPDCKTILKALGPAATLEEMMTACQGVGGPGHKARVLAEAMSQVTNSATIMMQRGNFRNQRKTVKCFNCGKEGHIAKNCRAPRKKGCWKCGKEGHQMKDCTERQANFLGKIWPSNKGRPGNFLQNRPEPTAPPEESFRFGEETTTPSQKQEPIDKEMYPLASLKSLFGNDPSSQMAPISPIETVPVKLKPGMDGPRVKQWPLTEEKIKALTAICEEMEKEGKITKIGPENPYNTPVFAIKKKDSTKWRKLVDFRELNKRTQDFWEVQLGIPHPAGLKKKKSVTVLAVGDAYFSVPLDEGFRKYTAFTIPSTNNETPGIRYQYNVLPQGWKGSPAIFQCSMTRILEPFRAKNPEIVIYQYMAALYVGSDLEIGQHRAKIEELREHLLKWGFTTPDKKHQKEPPFLWMGYELHPDKWTVQPIQLPEKDSWTVNDIQKLVGKLNWASQIYPGIKVRQLCKLLRGAKALTDIVPLTEEAELELAENREILKEPVHGVYYDPSKDLIAEIQKQGHDQWTYQIYQEPFKNLKTGKYAKMRTAHTNDVKQLTEAVQKIAMESIVIWGKTPKFRLPIQKETWETWWTDYWQATWIPEWEFVNTPPLVKLWYQLEKDPIAGVETFYVAGAANRETKLGKAGYVTDRGRQKIVSLTETTNQKTALQAIYLALQDSGSEVNIVTASQYALGIIQAQPDKSESELVNQIIEQLIKKERVYLSWVPAHKGIGGNEQVDKLVSSGIRKVLFLDGIDKAQEEHEKYHSNWRAMASDFNLPPVVAKEIVASCDQCQLKGEAMHGQVDCSPGIWQLACTHLEGKIILVAVHVASGYIEAEVIPAETGQETAYFILKLAGRWPVKVIHTANGSNFTSAAVKAACWWAGIQQEFGIPYNPQSQGVVASMNKELKKIIGQVRDQAEHLKTAVQMAVFIHNFKRKGGIGGYSAGERIIDIIATDIQTKELQKQIIKIQNFRVYYRDSRDPIWKGPAKLLWKGEGAVVIQDNSDIKVVPRRKVKIIKDYGKQMAGADCVAGRQDED

SEQ ID NO: 2 - protein, 1341 aa, Artificial Sequence
  Mos2.GagPol
MGARASILRGGKLDKWEKIRLRPGGKKHYMLKHLVWASRELERFALNPGLLETSEGCKQIIKQLQPALQTGTEELRSLFNTVATLYCVHAEIEVRDTKEALDKIEEEQNKSQQKTQQAKEADGKVSQNYPIVQNLQGQMVHQPISPRTLNAWVKVIEEKAFSPEVIPMFTALSEGATPQDLNTMLNTVGGHQAAMQMLKDTINEEAAEWDRLHPVHAGPVAPGQMREPRGSDIAGTTSNLQEQIAWMTSNPPIPVGDIYKRWIILGLNKIVRMYSPTSILDIKQGPKEPFRDYVDRFFKTLRAEQATQDVKNWMTDTLLVQNANPDCKTILRALGPGATLEEMMTACQGVGGPSHKARVLAEAMSQTNSTILMQRSNFKGSKRIVKCFNCGKEGHIARNCRAPRKKGCWKCGKEGHQMKDCTERQANFLGKIWPSHKGRPGNFLQSRPEPTAPPAESFRFEETTPAPKQEPKDREPLTSLRSLFGSDPLSQMAPISPIETVPVKLKPGMDGPKVKQWPLTEEKIKALVEICTEMEKEGKISKIGPENPYNTPIFAIKKKDSTKWRKLVDFRELNKRTQDFWEVQLGIPHPAGLKKKKSVTVLAVGDAYFSVPLDEDFRKYTAFTIPSINNETPGIRYQYNVLPQGWKGSPAIFQSSMTKILEPFRKQNPDIVIYQYMAALYVGSDLEIGQHRTKIEELRQHLLRWGFTTPDKKHQKEPPFLWMGYELHPDKWTVQPIVLPEKDSWTVNDIQKLVGKLNWASQIYAGIKVKQLCKLLRGTKALTEVVPLTEEAELELAENREILKEPVHGVYYDPSKDLIAEIQKQGQGQWTYQIYQEPFKNLKTGKYARMRGAHTNDVKQLTEAVQKIATESIVIWGKTPKFKLPIQKETWEAWWTEYWQATWIPEWEFVNTPPLVKLWYQLEKEPIVGAETFYVAGAANRETKLGKAGYVTDRGRQKVVSLTDTTNQKTALQAIHLALQDSGLEVNIVTASQYALGIIQAQPDKSESELVSQIIEQLIKKEKVYLAWVPAHKGIGGNEQVDKLVSRGIRKVLFLDGIDKAQEEHEKYHSNWRAMASEFNLPPIVAKEIVASCDKCQLKGEAIHGQVDCSPGIWQLACTHLEGKVILVAVHVASGYIEAEVIPAETGQETAYFLLKLAGRWPVKTIHTANGSNFTSATVKAACWWAGIKQEFGIPYNPQSQGVVASINKELKKIIGQVRDQAEHLKTAVQMAVFIHNFKRKGGIGEYSAGERIVDIIASDIQTKELQKQITKIQNFRVYYRDSRDPLWKGPAKLLWKGEGAVVIQDNSDIKVVPRRKAKIIRDYGKQMAGDDCVASRQDED

SEQ ID NO: 3 - protein, 685 aa, Artificial Sequence
  Mos1.Env
MRVTGIRKNYQHLWRWGTMLLGILMICSAAGKLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDDVRNVTNNATNTNSSWGEPMEKGEIKNCSFNITTSIRNKVQKQYALFYKLDVVPIDNDSNNTNYRLISCNTSVITQACPKVSFEPIPIHYCAPAGFAILKCNDKKFNGTGPCTNVSTVQCTHGIRPVVSTQLLLNGSLAEEEVVIRSENFTNNAKTIMVQLNVSVEINCTRPNNNTRKSIHIGPGRAFYTAGDIIGDIRQAHCNISRANWNNTLRQIVEKLGKQFGNNKTIVFNHSSGGDPEIVMHSFNCGGEFFYCNSTKLFNSTWTWNNSTWNNTKRSNDTEEHITLPCRIKQIINMWQEVGKAMYAPPIRGQIRCSSNITGLLLTRDGGNDTSGTEIFRPGGGDMRDNWRSELYKYKVVKIEPLGVAPTKAKRRVVQSEKSAVGIGAVFLGFLGAAGSTMGAASMTLTVQARLLLSGIVQQQNNLLRAIEAQQHLLQLTVWGIKQLQARVLAVERYLKDQQLLGIWGCSGKLICTTTVPWNASWSNKSLDKIWNNMTWMEWEREINNYTSLIYTLIEESQNQQEKNEQELLELDKWASLWNWFDISNWLW

SEQ ID NO: 4 - protein, 711 aa, Artificial Sequence
  Mos2S.Env
MRVRGMLRNWQQWWIWSSLGFWMLMIYSVMGNLWVTVYYGVPVWKDAKTTLFCASDAKAYEKEVHNVWATHACVPTDPNPQEIVLGNVTENFNMWKNDMVDQMHEDIISLWDASLEPCVKLTPLCVTLNCRNVRNVSSNGTYNIIHNETYKEMKNCSFNATTVVEDRKQKVHALFYRLDIVPLDENNSSEKSSENSSEYYRLINCNTSAITQACPKVSFDPIPIHYCAPAGYAILKCNNKTFNGTGPCNNVSTVQCTHGIKPVVSTQLLLNGSLAEEEIIIRSENLTNNAKTIIVHLNETVNITCTRPNNNTRKSIRIGPGQTFYATGDIIGDIRQAHCNLSRDGWNKTLQGVKKKLAEHFPNKTIKFAPHSGGDLEITTHTFNCRGEFFYCNTSNLFNESNIERNDSIITLPCRIKQIINMWQEVGRAIYAPPIAGNITCRSNITGLLLTRDGGSNNGVPNDTETFRPGGGDMRNNWRSELYKYKVVEVKPLGVAPTEAKRRVVEREKRAVGIGAVFLGILGAAGSTMGAASITLTVQARQLLSGIVQQQSNLLRAIEAQQHMLQLTVWGIKQLQTRVLAIERYLQDQQLLGLWGCSGKLICTTAVPWNTSWSNKSQTDIWDNMTWMQWDKEIGNYTGEIYRLLEESQNQQEKNEKDLLALDSWNNLWNWFSISKWLWYIKIFIMIVGGLIGLRIIFAVLSIVNRVRQGY

SEQ ID NO: 5 - protein, 679 aa, Artificial Sequence
  Clade C gp140 trimeric Env protein
AENLWVGNMWVTVYYGVPVWTDAKTTLFCASDTKAYDREVHNVWATHACVPTDPNPQEIVLENVTENFNMWKNDMVDQMHEDIISLWDQSLKPCVKLTPLCVTLHCTNATFKNNVTNDMNKEIRNCSFNTTTEIRDKKQQGYALFYRPDIVLLKENRNNSNNSEYILINCNASTITQACPKVNFDPIPIHYCAPAGYAILKCNNKTFSGKGPCNNVSTVQCTHGIKPVVSTQLLLNGSLAEKEIIIRSENLTDNVKTIIVHLNKSVEIVCTRPNNNTRKSMRIGPGQTFYATGDIIGDIRQAYCNISGSKWNETLKRVKEKLQENYNNNKTIKFAPSSGGDLEITTHSFNCRGEFFYCNTTRLFNNNATEDETITLPCRIKQIINMWQGVGRAMYAPPIAGNITCKSNITGLLLVRDGGEDNKTEEIFRPGGGNMKDNWRSELYKYKVIELKPLGIAPTGAKERVVEREERAVGIGAVFLGFLGAAGSTMGAASLTLTVQARQLLSSIVQQQSNLLRAIEAQQHMLQLTVWGIKQLQTRVLAIERYLKDQQLLGIWGCSGKLICTTNVPWNSSWSNKSQTDIWNNMTWMEWDREISNYTDTIYRLLEDSQTQQEKNEKDLLALDSWKNLWSWFDISNWLWYIKSRIEGRGSGGYIPEAPRDGQAYVRKDGEWVLLSTFL

SEQ ID NO: 6 - protein, 29 aa, Artificial Sequence
  example leader sequence
MRVRGIQRNCQHLWRWGTLILGMLMICSA

SEQ ID NO: 7 - DNA, 4056 bp, Artificial Sequence
  Mos1.GagPol exemplary nucleotide sequence
atgggagccagagccagcgtgctgtccggaggggagctggaccgctgggagaagatcaggctgaggcctggagggaagaagaagtacaggctgaagcacatcgtgtgggccagcagagagctggaacggtttgccgtgaaccctggcctgctggaaaccagcgagggctgtaggcagattctgggacagctgcagcccagcctgcagacaggcagcgaggaactgcggagcctgtacaacaccgtggccaccctgtactgcgtgcaccagcggatcgagatcaaggacaccaaagaagccctggaaaagatcgaggaagagcagaacaagagcaagaagaaagcccagcaggctgccgctgacacaggcaacagcagccaggtgtcccagaactaccccatcgtgcagaacatccagggacagatggtgcaccaggccatcagccctcggaccctgaacgcctgggtgaaggtggtggaggaaaaggccttcagccctgaggtgatccccatgttctctgccctgagcgagggagccacaccccaggacctgaacaccatgctgaacaccgtgggagggcaccaggctgccatgcagatgctgaaagagacaatcaacgaggaagctgccgagtgggacagggtccacccagtgcacgctggacctatcgctcctggccagatgagagagcccagaggcagcgatattgctggcaccacctccacactgcaggaacagatcggctggatgaccaacaaccctcccatccctgtgggagagatctacaagcggtggatcattctgggactgaacaagatcgtgcggatgtacagccctgtgagcatcctggacatcaggcagggacccaaagagcccttcagggactacgtggaccggttctacaagaccctgagagccgagcaggccagccaggacgtgaagaactggatgaccgagacactgctggtgcagaacgccaaccctgactgcaagaccatcctgaaagccctgggacctgctgccaccctggaagagatgatgacagcctgccagggagtgggaggacctggccacaaggccagggtgctggccgaggccatgagccaggtgaccaactctgccaccatcatgatgcagagaggcaacttccggaaccagagaaagaccgtgaagtgcttcaactgtggcaaagagggacacattgccaagaactgcagggctcccaggaagaaaggctgctggaagtgcggaaaagaaggccaccagatgaaggactgcaccgagaggcaggccaacttcctgggcaagatctggcctagcaacaagggcaggcctggcaacttcctgcagaacagacccgagcccaccgctcctcccgaggaaagcttccggtttggcgaggaaaccaccacccctagccagaagcaggaacccatcgacaaagagatgtaccctctggccagcctgaagagcctgttcggcaacgaccccagcagccagatggctcccatcagcccaatcgagacagtgcctgtgaagctgaagcctggcatggacggacccagggtgaagcagtggcctctgaccgaggaaaagatcaaagccctgacagccatctgcgaggaaatggaaaaagagggcaagatcaccaagatcggacccgagaacccctacaacacccctgtgttcgccatcaagaagaaagacagcaccaagtggaggaaactggtggacttcagagagctgaacaagcggacccaggacttctgggaggtgcagctgggcatccctcaccctgctggcctgaagaaaaagaaaagcgtgaccgtgctggctgtgggagatgcctacttcagcgtgcctctggacgagggcttccggaagtacacagccttcaccatccccagcaccaacaacgagacacctggcatcagataccagtacaacgtgctgcctcagggctggaaaggcagccctgccatcttccagtgcagcatgaccagaatcctggaacccttcagagccaagaaccctgagatcgtgatctaccagtatatggctgccctctacgtgggcagcgacctggaaatcggacagcacagagccaaaatcgaagaactccgcgagcacctgctgaagtggggattcaccacccctgacaagaagcaccagaaagagcctcccttcctgtggatgggctacgagctgcaccctgacaagtggaccgtgcagcccatccagctgccagagaaggactcctggaccgtgaacgacatccagaaactggtcggcaagctgaactgggccagccagatctaccctggcatcaaagtcagacagctgtgtaagctgctgaggggagccaaagcactgaccgacatcgtgcctctgacagaagaagccgagctggaactggccgagaacagagagatcctgaaagaacccgtgcacggagtgtactacgacccctccaaggacctgattgccgagatccagaaacagggacacgaccagtggacctaccagatctatcaggaacctttcaagaacctgaaaacaggcaagtacgccaagatgcggacagcccacaccaacgacgtgaagcagctgaccgaagccgtgcagaaaatcgccatggaaagcatcgtgatctggggaaagacacccaagttcaggctgcccatccagaaagagacatgggaaacctggtggaccgactactggcaggccacctggattcccgagtgggagttcgtgaacaccccacccctggtgaagctgtggtatcagctggaaaaggaccctatcgctggcgtggagacattctacgtggctggagctgccaacagagagacaaagctgggcaaggctggctacgtgaccgacagaggcagacagaaaatcgtgagcctgaccgaaaccaccaaccagaaaacagccctgcaggccatctatctggcactgcaggacagcggaagcgaggtgaacatcgtgacagccagccagtatgccctgggcatcatccaggcccagcctgacaagagcgagagcgagctggtgaaccagatcatcgagcagctgatcaagaaagaacgggtgtacctgagctgggtgccagcccacaagggcatcggagggaacgagcaggtggacaagctggtgtccagcggaatccggaaggtgctgttcctggacggcatcgataaagcccaggaagagcacgagaagtaccacagcaattggagagccatggccagcgacttcaacctgcctcccgtggtggccaaagaaatcgtggccagctgcgaccagtgccagctgaaaggcgaggccatgcacggacaggtggactgctcccctggcatctggcagctggcatgcacccacctggaaggcaagatcattctggtggccgtgcacgtggccagcggatacatcgaagccgaagtgatccctgccgagacagggcaggaaacagcctacttcatcctgaagctggctggcagatggcctgtgaaggtgatccacacagccaacggcagcaacttcacctctgctgccgtgaaggctgcctgttggtgggctggcattcagcaggaatttggcatcccctacaatccccagtctcagggagtggtggccagcatgaacaaagagctgaagaagatcatcggacaggtcagggatcaggccgagcacctgaaaactgccgtccagatggccgtgttcatccacaacttcaagcggaagggagggatcggagggtactctgctggcgagcggatcatcgacatcattgccaccgatatccagaccaaagagctgcagaaacagatcatcaagatccagaacttcagggtgtactacagggacagcagggaccccatctggaagggacctgccaagctgctgtggaaaggcgaaggagccgtcgtcatccaggacaacagcgacatcaaggtggtgcccagacggaaggtgaaaatcatcaaggactacggcaaacagatggctggagccgactgtgtcgctggcaggcaggacgaggactaatga

SEQ ID NO: 8 - DNA, 4029 bp, Artificial Sequence
  Mos2.GagPol exemplary nucleotide sequence
atgggagccagagccagcatcctgcgaggagggaagctggacaagtgggagaagatcaggctgaggcctggagggaagaaacactacatgctgaagcacctggtctgggccagcagagagctggaacggtttgccctcaatcctggcctgctggaaaccagcgagggctgcaagcagatcatcaagcagctgcagcctgccctgcagacaggcaccgaggaactgcggagcctgttcaacaccgtggccaccctgtactgcgtgcatgccgagatcgaagtgagggacaccaaagaagccctggacaagatcgaggaagagcagaacaagagccagcagaaaacccagcaggccaaagaagccgacggcaaggtctcccagaactaccccatcgtgcagaacctgcagggacagatggtgcaccagcccatcagccctcggacactgaatgcctgggtgaaggtgatcgaggaaaaggccttcagccctgaggtgatccccatgttcacagccctgagcgagggagccacaccccaggacctgaacaccatgctgaacaccgtgggagggcaccaggctgccatgcagatgctgaaggacaccatcaacgaggaagctgccgagtgggacaggctgcaccctgtgcacgctggacctgtggctcctggccagatgagagagcccagaggcagcgatattgctggcaccacctccaatctgcaggaacagatcgcctggatgaccagcaaccctcccatccctgtgggagacatctacaagcggtggatcatcctgggactgaacaagatcgtgcggatgtacagccctacctccatcctggacatcaagcagggacccaaagagcctttcagggactacgtggaccggttcttcaagaccctgagagccgagcaggccacccaggacgtgaagaactggatgaccgacaccctgctggtgcagaacgccaaccctgactgcaagaccatcctgagagccctgggacctggagccaccctggaagagatgatgacagcctgccagggagtgggaggaccctctcacaaggctagggtgctggccgaggccatgagccagaccaacagcaccatcctgatgcagcggagcaacttcaagggcagcaagcggatcgtgaagtgcttcaactgtggcaaagagggacacattgccagaaactgtagggcacccaggaagaaaggctgctggaagtgcggaaaagaaggccaccagatgaaggactgcaccgagaggcaggccaacttcctgggcaagatctggcctagccacaagggcagacctggcaacttcctgcagagcagacccgagcccaccgctcctccagccgagagcttccggttcgaggaaaccacccctgctcccaagcaggaacctaaggacagagagcctctgaccagcctgagaagcctgttcggcagcgaccctctgagccagatggctcccatctcccctatcgagacagtgcctgtgaagctgaagcctggcatggacggacccaaggtgaaacagtggcctctgaccgaggaaaagatcaaagccctggtggagatctgtaccgagatggaaaaagagggcaagatcagcaagatcggacccgagaacccctacaacacccctatcttcgccatcaagaagaaagacagcaccaagtggaggaaactggtggacttcagagagctgaacaagcggacccaggacttctgggaggtgcagctgggcatccctcaccctgctggcctgaagaaaaagaaaagcgtgaccgtgctggccgtgggagatgcctacttcagcgtgcctctggacgaggacttcagaaagtacacagccttcaccatccccagcatcaacaacgagacacctggcatcagataccagtacaacgtgctgcctcagggatggaagggctctcctgcaatcttccagagcagcatgaccaagatcctggaacccttccggaagcagaaccctgacatcgtgatctaccagtacatggcagccctgtacgtcggcagcgacctggaaatcggacagcaccggaccaagatcgaagaactcaggcagcacctgctgcggtggggattcaccacccctgacaagaagcaccagaaagagcctcccttcctgtggatgggctacgagctgcacccagacaagtggaccgtgcagcccatcgtgctgcctgagaaggactcctggaccgtgaacgacatccagaaactggtcggcaagctgaactgggccagccagatctacgctggcatcaaagtgaagcagctgtgtaagctcctgagaggcaccaaagccctgaccgaggtggtgccactgacagaggaagccgagctggaactggccgagaacagagagatcctgaaagaacccgtgcacggagtgtactacgaccccagcaaggacctgattgccgagatccagaagcagggacagggacagtggacctaccagatctaccaggaacccttcaagaacctgaaaacaggcaagtacgccaggatgaggggagcccacaccaacgacgtcaaacagctgaccgaagccgtgcagaagatcgccaccgagagcatcgtgatttggggaaagacacccaagttcaagctgcccatccagaaagagacatgggaggcctggtggaccgagtactggcaggccacctggattcccgagtgggagttcgtgaacaccccacccctggtgaagctgtggtatcagctggaaaaagaacccatcgtgggagccgagacattctacgtggctggagctgccaacagagagacaaagctgggcaaggctggctacgtgaccgacagaggcaggcagaaagtggtgtccctgaccgataccaccaaccagaaaacagccctgcaggccatccacctggctctgcaggactctggcctggaagtgaacatcgtgacagccagccagtatgccctgggcatcattcaggcacagcctgacaagagcgagagcgagctggtgtctcagatcattgagcagctgatcaagaaagaaaaggtgtacctggcctgggtgccagcccacaaggggatcggagggaacgagcaggtggacaagctggtgtccaggggcatccggaaggtgctgtttctggacggcatcgacaaagcccaggaagagcacgagaagtaccacagcaattggagagccatggccagcgagttcaacctgcctcccatcgtggccaaagaaatcgtggcctcttgcgacaagtgccagctgaaaggcgaggccattcacggacaggtggactgcagcccaggcatctggcagctggcctgcacccacctggaaggcaaggtgatcctggtggccgtgcacgtggcctctggatacatcgaagccgaagtgatccctgccgagacaggccaggaaacagcctacttcctgctgaagctggctggcaggtggcctgtgaaaaccatccacacagccaacggcagcaacttcacctctgccaccgtgaaggctgcctgttggtgggctggcattaagcaggaatttggcatcccctacaaccctcagtctcagggagtggtggcctccatcaacaaagagctgaagaagatcatcggacaggtcagggatcaggccgagcatctgaaaacagccgtccagatggccgtgttcatccacaacttcaagcggaagggagggatcggagagtactctgctggcgagaggatcgtggacattatcgccagcgatatccagaccaaagaactgcagaagcagatcacaaagatccagaacttcagggtgtactacagggacagcagagatcccctgtggaagggacctgccaagctgctgtggaaaggcgaaggagccgtcgtcatccaggacaacagcgacatcaaggtggtgcccagacggaaggccaagatcatcagagactacggcaaacagatggctggcgacgactgcgtcgcctctaggcaggacgaggactaatga

SEQ ID NO: 9 - DNA, 2061 bp, Artificial Sequence
  Mos1.Env exemplary nucleotide sequence
atgcgggtgaccggcatccggaagaactaccagcacctgtggcggtggggcaccatgctgctgggcatcctgatgatttgctctgccgccggaaagctgtgggtgaccgtgtactacggcgtgcccgtgtggaaagaggccaccaccaccctgttctgcgccagcgacgccaaggcctacgacaccgaggtgcacaacgtgtgggccacccacgcctgcgtgcccaccgaccccaacccccaggaagtggtcctggaaaacgtgaccgagaacttcaacatgtggaagaacaacatggtggagcagatgcacgaggacatcatcagcctgtgggaccagagcctgaagccctgcgtgaagctgacccccctgtgcgtgaccctgaactgcaccgacgacgtgcggaacgtgaccaacaacgccaccaacaccaacagcagctggggcgagcctatggaaaagggcgagatcaagaactgcagcttcaacatcaccacctccatccggaacaaggtgcagaagcagtacgccctgttctacaagctggacgtggtgcccatcgacaacgacagcaacaacaccaactaccggctgatcagctgcaacaccagcgtgatcacccaggcctgccccaaggtgtccttcgagcccatccccatccactactgcgcccctgccggcttcgccatcctgaagtgcaacgacaagaagttcaacggcaccggcccctgcaccaacgtgagcaccgtgcagtgcacccacggcatccggcccgtggtgtccacccagctgctgctgaacggcagcctggccgaggaagaggtggtgatcagaagcgagaatttcaccaacaatgccaagaccatcatggtgcagctgaacgtgagcgtggagatcaactgcacccggcccaacaacaacacccggaagagcatccacatcggccctggcagggccttctacacagccggcgacatcatcggcgacatccggcaggcccactgcaacatcagccgggccaactggaacaacaccctgcggcagatcgtggagaagctgggcaagcagttcggcaacaacaagaccatcgtgttcaaccacagcagcggcggagaccccgagatcgtgatgcacagcttcaactgtggcggcgagttcttctactgcaacagcaccaagctgttcaacagcacctggacctggaacaactccacctggaataacaccaagcggagcaacgacaccgaagagcacatcaccctgccctgccggatcaagcagattatcaatatgtggcaggaggtcggcaaggccatgtacgcccctcccatccggggccagatccggtgcagcagcaacatcaccggcctgctgctgacccgggacggcggcaacgataccagcggcaccgagatcttccggcctggcggcggagatatgcgggacaactggcggagcgagctgtacaagtacaaggtggtgaagatcgagcccctgggcgtggctcccaccaaggccaagcggcgggtggtgcagagcgagaagagcgccgtgggcatcggcgccgtgtttctgggcttcctgggagccgccggaagcaccatgggagccgccagcatgaccctgaccgtgcaggcccggctgctgctgtccggcatcgtgcagcagcagaacaacctgctccgggccatcgaggcccagcagcacctgctgcagctgaccgtgtggggcatcaagcagctgcaggccagggtgctggccgtggagagatacctgaaggatcagcagctcctggggatctggggctgcagcggcaagctgatctgcaccaccaccgtgccctggaacgccagctggtccaacaagagcctggacaagatctggaacaatatgacctggatggaatgggagcgcgagatcaacaattacaccagcctgatctacaccctgatcgaggaaagccagaaccagcaggaaaagaacgagcaggaactgctggaactggacaagtgggccagcctgtggaactggttcgacatcagcaactggctgtggtaatga

SEQ ID NO: 10 - DNA, 2139 bp, Artificial Sequence
  Mos2S.Env exemplary nucleotide sequence
atgagagtgcggggcatgctgagaaactggcagcagtggtggatctggtccagcctgggcttctggatgctgatgatctacagcgtgatgggcaacctgtgggtcaccgtgtactacggcgtgcccgtgtggaaggacgccaagaccaccctgttttgcgcctccgatgccaaggcctacgagaaagaggtgcacaacgtctgggccacccacgcctgtgtgcccaccgaccccaatccccaggaaatcgtcctgggcaacgtgaccgagaacttcaacatgtggaagaacgacatggtcgatcagatgcacgaggacatcatctccctgtgggacgcctccctggaaccctgcgtgaagctgacccctctgtgcgtgaccctgaactgccggaacgtgcgcaacgtgtccagcaacggcacctacaacatcatccacaacgagacatacaaagagatgaagaactgcagcttcaacgctaccaccgtggtcgaggaccggaagcagaaggtgcacgccctgttctaccggctggacatcgtgcccctggacgagaacaacagcagcgagaagtcctccgagaacagctccgagtactacagactgatcaactgcaacaccagcgccatcacccaggcctgccccaaggtgtccttcgaccctatccccatccactactgcgcccctgccggctacgccatcctgaagtgcaacaacaagaccttcaatggcaccggcccctgcaacaatgtgtccaccgtgcagtgcacccacggcatcaagcccgtggtgtctacccagctgctgctgaacggcagcctggccgaggaagagatcattatcagaagcgagaacctgaccaacaacgccaaaaccatcatcgtccacctgaacgaaaccgtgaacatcacctgtacccggcctaacaacaacacccggaagtccatccggatcggccctggccagaccttttacgccaccggcgatattatcggcgacatccggcaggcccactgcaatctgagccgggacggctggaacaagacactgcagggcgtcaagaagaagctggccgaacacttccctaacaagactatcaagttcgcccctcactctggcggcgacctggaaatcaccacccacaccttcaactgtcggggcgagttcttctactgcaatacctccaacctgttcaacgagagcaacatcgagcggaacgacagcatcatcacactgccttgccggatcaagcagattatcaatatgtggcaggaagtgggcagagccatctacgcccctccaatcgccggcaacatcacatgccggtccaatatcaccggcctgctgctcaccagagatggcggctccaacaatggcgtgccaaacgacaccgagacattcagacccggcggaggcgacatgcggaacaattggcggagcgagctgtacaagtacaaggtggtggaagtgaagcccctgggcgtggcccctaccgaggccaagagaagagtggtcgaacgcgagaagcgggccgtgggaatcggagccgtgtttctgggaatcctgggagccgctggctctaccatgggcgctgcctctatcaccctgacagtgcaggccagacagctgctcagcggcatcgtgcagcagcagagcaacctgctgagagccattgaggcccagcagcacatgctgcagctgaccgtgtggggcattaagcagctccagacacgggtgctggccatcgagagatacctgcaggatcagcagctcctgggcctgtggggctgtagcggcaagctgatctgtaccaccgccgtgccctggaatacctcttggagcaacaagagccagaccgacatctgggacaacatgacctggatgcagtgggacaaagaaatcggcaactataccggcgagatctatagactgctggaagagtcccagaaccagcaggaaaagaacgagaaggacctgctggccctggattcttggaacaatctgtggaactggttcagcatctccaagtggctgtggtacatcaagatcttcatcatgatcgtgggcggcctgatcggcctgcggatcatctttgccgtgctgagcatcgtgaaccgcgtgcggcagggctactgataa

SEQ ID NO: 11 - DNA, 2130 bp, Artificial Sequence
  exemplary nucleic acid sequence encoding Clade C gp140 trimeric Env protein, including leader sequence
atgagagtgcggggcatccagcggaactgccagcatctgtggcgctggggcaccctgatcctgggcatgctgatgatctgctctgccgccgagaacctgtgggtcggaaatatgtgggtcaccgtgtactacggcgtgcccgtgtggaccgacgccaagaccaccctgttctgcgccagcgacaccaaggcctacgaccgcgaggtgcacaacgtgtgggccacccacgcttgtgtgcctaccgaccccaacccccaggaaatcgtcctggaaaacgtgaccgagaacttcaacatgtggaagaacgacatggtggaccagatgcacgaggacatcatcagcctgtgggaccagagcctgaagccctgcgtgaagctgacacccctgtgcgtgaccctgcactgcaccaacgccaccttcaagaacaacgtgaccaacgacatgaacaaagagatccggaactgcagcttcaacaccaccaccgagatccgggacaagaagcagcagggctacgccctgttctaccggcccgacatcgtgctgctgaaagagaacagaaacaacagcaacaactccgagtacatcctgatcaactgcaacgccagcaccatcacccaggcctgccccaaagtgaacttcgaccctatccccatccactactgcgcccctgccggctacgccatcctgaagtgcaacaacaagaccttcagcggcaagggcccctgcaacaacgtgtccaccgtgcagtgcacccacggcatcaagcccgtggtgtccacccagctgctgctgaatggcagcctggccgagaaagagatcatcatcagaagcgagaacctgaccgacaacgtcaagaccatcatcgtgcacctgaacaagagcgtggaaatcgtgtgcacccggcccaacaacaacaccagaaagagcatgcggatcggccctggccagaccttctacgccaccggcgacatcatcggcgacatccggcaggcctactgcaacatcagcggcagcaagtggaacgagacactgaagagagtgaaagagaagctgcaggaaaactataacaacaacaaaaccatcaagttcgcccccagctctggcggcgacctggaaatcaccacccacagcttcaactgcagaggcgagttcttctactgcaataccacccggctgttcaacaacaacgccaccgaggacgagacaatcaccctgccctgccggatcaagcagatcatcaatatgtggcagggcgtgggcagagctatgtacgcccctcctatcgccggcaacatcacatgcaagagcaacatcaccggcctgctgctcgtgcgggacggcggcgaggataacaagaccgaggaaatcttcagacccggcggaggcaacatgaaggacaactggcggagcgagctgtacaagtacaaagtgatcgagctgaagcccctgggaatcgcccctaccggagccaaggaaagagtggtggaacgcgaggagcgggccgtgggaatcggcgccgtgttcctgggctttctgggagccgccggaagcacaatgggcgctgccagcctgaccctgaccgtgcaggccagacagctgctgagcagcatcgtgcagcagcagagcaacctcctgagagccatcgaggcccagcagcacatgctgcagctgaccgtgtggggcatcaagcagctgcagacccgggtgctggccatcgagagatacctgaaggaccagcagctcctgggcatctggggctgcagcggcaagctgatctgcaccaccaacgtgccctggaacagcagctggtccaacaagagccagaccgacatctggaacaacatgacctggatggaatgggaccgcgagatcagcaactacaccgacaccatctaccggctgctggaagatagccagacccagcaggaaaagaacgagaaggacctgctggccctggacagctggaagaatctgtggtcttggtttgacatcagcaactggctgtggtacatcaagagccggatcgagggcagaggcagcggcggctacatccctgaggcccctagagatggccaggcctacgtgcggaaggacggcgaatgggtgctgctgtccaccttcctgtgataa